SEQUENCE LISTING (32 sequences)

SEQ ID NO: 1 - skipped

SEQ ID NO: 2 - protein, 5 aa, synthetic construct
  REGION 1..5: note = Description of sequence: Spacer
GGGGS

SEQ ID NO: 3 - protein, 4 aa, synthetic construct
  REGION 1..4: note = Description of sequence: Spacer
GGSG

SEQ ID NO: 4 - protein, 4 aa, synthetic construct
  REGION 1..4: note = Description of sequence: Spacer
SGGG

SEQ ID NO: 5 - protein, 10 aa, synthetic construct
  REGION 1..10: note = Description of sequence: Spacer
GSGSGSGSGS

SEQ ID NO: 6 - protein, 12 aa, synthetic construct
  REGION 1..12: note = Description of sequence: Spacer
GSGSGSGSGSGS

SEQ ID NO: 7 - protein, 6 aa, synthetic construct
  REGION 1..6: note = Description of sequence: Spacer
GGSGGS

SEQ ID NO: 8 - protein, 9 aa, synthetic construct
  REGION 1..9: note = Description of sequence: Spacer
GGSGGSGGS

SEQ ID NO: 9 - protein, 12 aa, synthetic construct
  REGION 1..12: note = Description of sequence: Spacer
GGSGGSGGSGGS

SEQ ID NO: 10 - protein, 8 aa, synthetic construct
  REGION 1..8: note = Description of sequence: Spacer
GGSGGGSG

SEQ ID NO: 11 - protein, 12 aa, synthetic construct
  REGION 1..12: note = Description of sequence: Spacer
GGSGGGSGGGSG

SEQ ID NO: 12 - protein, 15 aa, synthetic construct
  REGION 1..15: note = Description of sequence: Spacer
GGGGSGGGGSGGGGS

SEQ ID NO: 13 - protein, 4 aa, synthetic construct
  REGION 1..4: note = Description of sequence: Spacer
GSGS

SEQ ID NO: 14 - protein, 6 aa, synthetic construct
  REGION 1..6: note = Description of sequence: Spacer
GSGSGS

SEQ ID NO: 15 - protein, 8 aa, synthetic construct
  REGION 1..8: note = Description of sequence: Spacer
GSGSGSGS

SEQ ID NO: 16 - protein, 1678 aa, synthetic construct
  REGION 1..1678: note = Description of sequence: Construct of HTN-BRD4
  REGION 1367..1540: note = The 1051-1224 fragment, where most O-GlcNAc modification occurs
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTEDLYFQSDNAIASEFMSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 17 - DNA, 33 bp, synthetic construct
  misc_feature 1..33: note = Description of sequence: Primer; Primer No: 1, Name: 4TPR(327-1046)-fOGT fwd
  source 1..33: mol_type = other DNA
cacagcagactctctgaataacctagccaatat

SEQ ID NO: 18 - DNA, 33 bp, synthetic construct
  misc_feature 1..33: note = Description of sequence: Primer; Primer No: 2, Name: 4TPR(327-1046)-fOGT rev
  source 1..33: mol_type = other DNA
ttcagagagtctgctgtgctgtcggccacgttg

SEQ ID NO: 19 - DNA, 33 bp, synthetic construct
  misc_feature 1..33: note = Description of sequence: Primer; Primer No: 3, Name: 2TPR(395-1046)-fOGT fwd
  source 1..33: mol_type = other DNA
acagctgatgcctactctaatatgggaaacact

SEQ ID NO: 20 - DNA, 33 bp, synthetic construct
  misc_feature 1..33: note = Description of sequence: Primer; Primer No: 4, Name: 2TPR(395-1046)-fOGT rev
  source 1..33: mol_type = other DNA
agagtaggcatcagctgtgctgtcggccacgtt

SEQ ID NO: 21 - DNA, 33 bp, synthetic construct
  misc_feature 1..33: note = Description of sequence: Primer; Primer No: 5, Name: 0TPR(463-1046)-fOGT fwd
  source 1..33: mol_type = other DNA
acagcacacctgatgcttattgtaacttggctc

SEQ ID NO: 22 - DNA, 33 bp, synthetic construct
  misc_feature 1..33: note = Description of sequence: Primer; Primer No: 6, Name: 0TPR(463-1046)-fOGT rev
  source 1..33: mol_type = other DNA
aagcatcaggtgtgctgtcggccacgttgccca

SEQ ID NO: 23 - DNA, 33 bp, synthetic construct
  misc_feature 1..33: note = Description of sequence: Primer; Primer No: 7, Name: HTN-BRD4 ?1051-1224 rev fwd
  source 1..33: mol_type = other DNA
caccacaaggagcagttccgccgcgccgctcgg

SEQ ID NO: 24 - DNA, 33 bp, synthetic construct
  misc_feature 1..33: note = Description of sequence: Primer; Primer No: 8, Name: HTN-BRD4 ?1051-1224 rev
  source 1..33: mol_type = other DNA
gaactgctccttgtggtgccggggtgaatggtg

SEQ ID NO: 25 - DNA, 34 bp, synthetic construct
  misc_feature 1..34: note = Description of sequence: Primer; Primer No: 9, Name: HTN-BRD4 S1051A/ S1055A fwd
  source 1..34: mol_type = other DNA
ggcggacccctacgcaaccggtcacctccgcgaa

SEQ ID NO: 26 - DNA, 34 bp, synthetic construct
  misc_feature 1..34: note = Description of sequence: Primer; Primer No: 10, Name: HTN-BRD4 S1051A/ S1055A rev
  source 1..34: mol_type = other DNA
ttgcgtaggggtccgccttgtggtgccggggtga

SEQ ID NO: 27 - DNA, 41 bp, synthetic construct
  misc_feature 1..41: note = Description of sequence: Primer; Primer No: 11, Name: HTN-BRD4 1201A/1204A/1211A fwd
  source 1..41: mol_type = other DNA
ccctagtgcagaagcatccggccaccccctcctccacagcc

SEQ ID NO: 28 - DNA, 49 bp, synthetic construct
  misc_feature 1..49: note = Description of sequence: Primer; Primer No: 12, Name: HTN-BRD4 1201A/1204A/1211A rev
  source 1..49: mol_type = other DNA
atgcttctgcactagggcggcccaggcgcccatgttcttgattttcagg

SEQ ID NO: 29 - DNA, 33 bp, synthetic construct
  misc_feature 1..33: note = Description of sequence: Primer; Primer No: 13, Name: HTN-CK2? S347A fwd
  source 1..33: mol_type = other DNA
tcgccagcgccaatatgatgtcagggatttctt

SEQ ID NO: 30 - DNA, 34 bp, synthetic construct
  misc_feature 1..34: note = Description of sequence: Primer; Primer No: 14, Name: HTN-CK2? S347A rev
  source 1..34: mol_type = other DNA
tcatattggcgctggcgacgggcgtactgccccc

SEQ ID NO: 31 - DNA, 33 bp, synthetic construct
  misc_feature 1..33: note = Description of sequence: Primer; Primer No: 15, Name: HTN-CK2? S347A/S348A fwd
  source 1..33: mol_type = other DNA
tcgccgccgccaatatgatgtcagggatttctt

SEQ ID NO: 32 - DNA, 34 bp, synthetic construct
  misc_feature 1..34: note = Description of sequence: Primer; Primer No: 16, Name: HTN-CK2? S347A/S348A rev
  source 1..34: mol_type = other DNA
tcatattggcggcggcgacgggcgtactgccccc

(19) United States
(12) Patent Application Publication
(10) Pub. No.: US 2025/0035641 A1
(43) Pub. Date: Jan. 30, 2025

(54) COMPOSITIONS AND METHODS RELATED TO PROTEIN LABELING

(71) Applicant: The Chinese University of Hong Kong, Shatin (CN)

(72) Inventors: Wai-Lung NG, Shatin, N.T., Hong Kong SAR (CN); Bowen MA, Shatin, N.T., Hong Kong SAR (CN)

(21) Appl. No.: 18/667,437

(22) Filed: May 17, 2024

Related U.S. Application Data
(60) Provisional application No. 63/467,875, filed on May 19, 2023.

(51) Int. Cl. G01N 33/68

(52) U.S. Cl. CPC G01N 33/6848; G01N 33/6845; G01N 2440/38; G01N 2458/00; G01N 2570/00

(57) ABSTRACT

The disclosure provides compositions and methods for labeling a protein with a glycan by a glycan transferase by using a proximity inducing ligand.

### CROSS-REFERENCE TO RELATED APPLICATION

The present application claims priority to U.S. Provisional Patent Application No. 63/467,875, filed on May 19, 2023, the disclosure of which is incorporated herein by reference in its entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Aug. 15, 2024, is entitled “080015-1442207-039310US_ST26.xml”, and is 42,589 bytes in size.

### BACKGROUND OF THE INVENTION

Protein O-linked β-N-acetylglucosamine modification (O-GlcNAcylation) plays a significant role in the regulation of transcription, metabolism, cell signalling, protein stability, and nucleocytoplasmic trafficking. Increasing amount of research uncovered that abnormal regulation of O-GlcNAcylation related to many pathological disorders, including cancer, neurodegenerative diseases, cardiovascular diseases, autoimmune diseases, and diabetes.

### BRIEF SUMMARY OF THE INVENTION

In one aspect, the disclosure provides a method of labeling a protein of interest (POI) with a glycan, comprising: (a) contacting, in a sample, a first fusion protein comprising a glycan transferase fused to a first proximity inducing protein, and a second fusion protein comprising the POI fused to a second proximity inducing protein, with a proximity inducing ligand; and (b) incubating the sample for a duration sufficient for the first and second proximity inducing proteins to both to bind to the proximity inducing ligand and for the glycan transferase to label the POI with a glycan.

In some embodiments, the glycan transferase is a O-GlcNAc transferase, a polypeptide N-Acetylgalactosaminyltransferase 14 (GALNT14), or a glucuronosyltransferase.

In some embodiments, the first proximity inducing protein is a FKBP12^{F36V}. In some embodiments, the second proximity inducing protein is a Halotag. In some embodiments, the second proximity inducing protein is fused to the N-terminus or the C-terminus of the POI.

In some embodiments, the proximity inducing ligand comprises a first portion that is recognized and bound by the first proximity inducing protein and a second portion that is recognized and bound by the second proximity protein.

In some embodiments, the first portion is AP1867. In some embodiments, the second portion is a haloalkane, such as a chloroalkane.

In some embodiments, the proximity inducing ligand further comprises a chemical linker between the first portion and the second portion. In some embodiments, the chemical linker is a PEG linker or an aliphatic linker, such as a C2-C8 linker, e.g., C2, C3, C4, CS, C6, C7, or C8 linker.

In some embodiments, the proximity inducing ligand is:

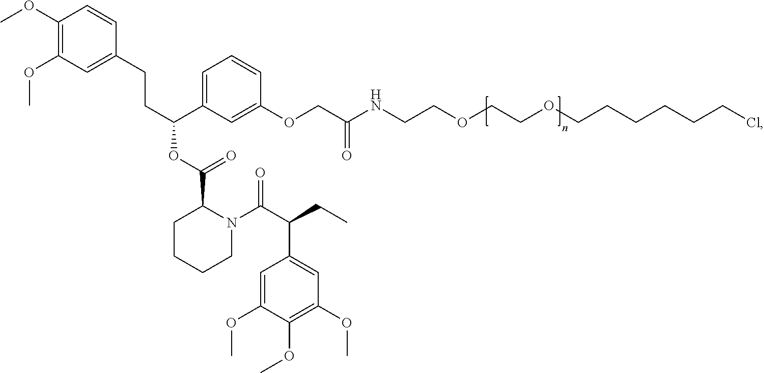

wherein n is an integer between 1 and 8.

In some embodiments, n is 1, 4, or 6.

In some embodiments, the method further comprises the step (c) of contacting the sample with a chase ligand that is recognized and bound by the second proximity protein. In some embodiments, the chase ligand comprises a detectable portion and a portion that is recognized and bound by the second proximity protein. In some embodiments, the chase ligand is:

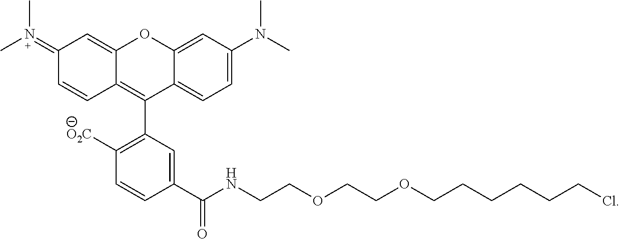

In another aspect, the disclosure features a method of labeling a protein of interest (POI) with a glycan, comprising: (a) contacting, in a sample, a fusion protein comprising a glycan transferase fused to a proximity inducing protein, and the POI, with a proximity inducing ligand; and (b) incubating the sample for a duration sufficient for the fusion protein and the POI to both to bind to the proximity inducing ligand and for the glycan transferase to label the POI with a glycan.

In some embodiments, the glycan transferase is a O-GlcNAc transferase, a polypeptide N-Acetylgalactosaminyltransferase 14 (GALNT14), or a glucuronosyltransferase.

In some embodiments, the proximity inducing protein is a FKBP12^{F36V}.

In some embodiments, the proximity inducing ligand comprises a first portion that is recognized and bound by the proximity inducing protein and a second portion that is recognized and bound by the POI. In some embodiments, the first portion is AP1867. In some embodiments, the second portion is an optionally substituted BET bromodomain inhibitor (JQ1), an optionally substituted EZH2 inhibitor, an optionally substituted BRD inhibitor, or an optionally substituted kinase inhibitor. In some embodiments, the second portion is an optionally substituted BET bromodomain inhibitor (JQ1).

In some embodiments, the proximity inducing ligand further comprises a chemical linker between the first portion and the second portion. In some embodiments, the chemical linker is a PEG linker or an aliphatic linker, such as a C2-C8 linker, e.g., C2, C3, C4. C5, C6, C7, or C8 linker.

In some embodiments, the proximity inducing ligand is:

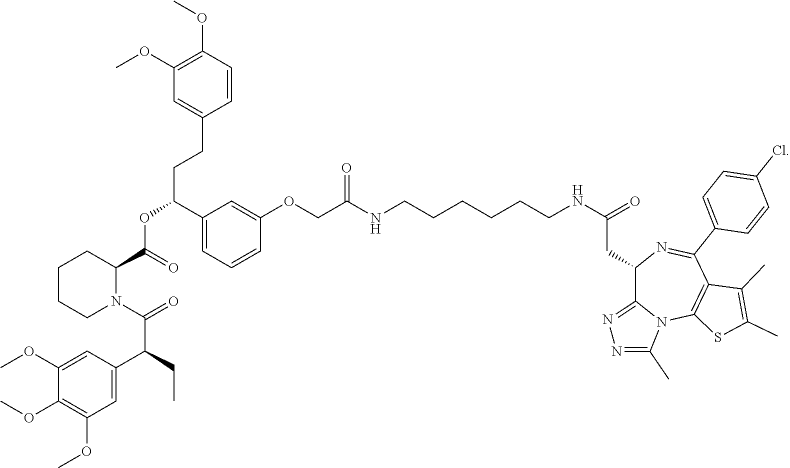

In some embodiments, the method further comprises the step (c) of contacting the sample with a chase ligand that is recognized and bound by the POI. In some embodiments, the chase ligand comprises a detectable portion and a portion that is recognized and bound by the POI.

In some embodiments of the methods, the sample is incubated for at least 30 minutes in step (b). In some embodiments, the sample is incubated for at least 2 hours in step (b).

### BRIEF DESCRIPTION OF THE DRAWINGS

FIGS. 1A-1D: General concept of OGTAC technology and target engagement study of OGTAC molecules. (A), Conceptual scheme of OGTACs, which induce POI specific O-GlcNAcylation by recruiting FKBP12^{F36V}-OGT to Halotag-fused POIs. (B) Chemical structure of OGTAC-1,2,3, and rhodamine ligand for the pulse-chase experiment. (C, D) confirmation of OGTAC1,2,3 target engagement in cells. HEK293T cells were transient transfected with plasmids HTN-BRD4:fOGT at 20:1 ratio. (C) In pulse-chase assay, OGTAC-1,2,3 (5 μM) or vehicle were added to cells for increasing period of time, followed by the replacement of rhodamine ligand to label any remaining HTN-BRD4 proteins that had not been engaged by OGTACs. Then, cells were lysed and submitted to SDS-PAGE for in gel fluorescence in rhodamine channel and immunoblotted using Halotag antibody to verify equal loading. (D) After 4 h treatment of OGTAC-1,2,3, intact cell CETSA was conducted to verify their direct binding to fOGT. Data in (c, d) represent as mean±s.d. of n=3 replicates.

FIGS. 2A-2C: OGTAC-1 induced dose-dependent, rapid, targeted O-GlcNAcylation of HTN-BRD4 by recruiting fOGT in cells. (A), OGTAC-1 showed best O-GlcNAcylation inducing potency among three OGTACs at 5 μM. (B), Dose-dependent O-GlcNAcylation profile of HTN-BRD4 by OGTAC-1 treatment. HEK293T cells expressing fOGT:HTN-BRD4=1:20 were treated with increasing concentration of OGTAC-1 for 4 h. Then, the O-GlcNAcylation level of HTN-BRD4 was assessed by immunoblot after IP using BRD4 antibody. Co-IP of fOGT was also observed in an OGTAC-1 dose-dependent manner. (C) OGTAC-1 (640 nM) O-GlcNAcylation inducing effects on HTN-BRD4 over indicated time course. For (A) and (B), the right panels show the quantifications of the immunoblot signal of RL2 relative to HTN-BRD4 and Co-IPed fOGT relative to IPed BRD4, as the mean±s.e.m. of n=3 biologically independent experiments. Notably, the marginal divergence between the red and black curves after 4 h suggests diminished effect of OGTAC-1. Statistical significance of (A) and (B) was calculated with ordinary one-way ANOVA; that of (C) was calculated with unpaired multiple t-test *p<0.05; **p<0.01.

FIGS. 3A-3E: OGTAC technology extended to other POIs. (A), IP-WB method showed OGTAC induced HTN-CK2α O-GlcNAcylation. (B), Mass-shift assay to validate the OGTAC-1 induced specific HTN-CK2α O-GlcNAcylation. (C), OGTAC-1 induced dose-dependent O-GlcNAcylation of HTN-CK2α in cells. HEK293T cells were transfected with HTN-CK2α:fOGT=1:0.01 for 24 h, followed by treatment with increasing concentration of OGTAC-1 for 8 h. Transfection with HTN-CK2α:fOGT=1:0.2 was set as positive control. (D), OGTAC-1 induced physiologically relevant S347 O-GlcNAcylation in HTN-CK2α. ‘++’ denotes the HTN-CK2α:fOGT=1:0.2. while ‘+’ denotes the HTN-CK2α:fOGT=1:0.01. (E) Immunoblot analysis of OGTAC-1 mediated O-GlcNAcylation when co-treated with binding competitors. All quantifications are shown as mean±s.e.m. of 3 independent biological repeats. Statistical significance for (B) was calculated with two-tailed Student's t-test. and remaining significance were calculated with ordinary one-way ANOVA comparing DMSO- to OGTAC-1-treated samples. ns, p≥0.05; *p<0.05; **p<0.01; ***p<0.001.

FIG. 4A-4E: OGTAC-4 induces BRD4 O-GlcNAcylation by recruiting fOGT in cells; (A), Conceptual scheme of OGTAC-4, which induces BRD4-specific O-GlcNAcylation by recruiting FKBP12^{F36V}-OGT to BRD4 through JQ1 motif. (B), Chemical structure of OGTAC-4. (C), Dose-dependent O-GlcNAcylation profile of HTN-BRD4 by OGTAC-4 treatment. HEK293T cells expressing HTN-BRD4:fOGT=1:0.05 were treated with an increasing concentration of OGTAC-4 for 4 h. Then, the O-GlcNAcylation level of HTN-BRD4 was assessed by immunoblot after IP using BRD4 antibody. Co-IP of fOGT was also observed in an OGTAC-4 dose-dependent manner. (D), OGTAC-4 (640 nM) O-GlcNAcylation-inducing effects on HTN-BRD4 over indicated time course. (E), OGTAC-1 and OGTAC-4 induced HTN-BRD4 O-GlcNAcylation in C-terminal region (S1367-F1224). ‘+’ denotes the HTN-BRD4:fOGT=1:0.05. The rectangles with blue stripes represent the truncated fragment used for validating O-GlcNAcylation sites HAT, histone acetyltransferase catalytic domain; Statistical significance was calculated with ordinary one-way ANOVA comparing DMSO- to OGTAC-4-treated samples. The quantifications of the immunoblot shows the signal of RL2 relative to HTN-BRD4 and Co-IPed fOGT relative to IPed BRD4, as the mean±s.e.m. of n=3 biologically independent experiments. Statistical significance for (C) was calculated with ordinary one-way ANOVA; for (D) was calculated with unpaired multiple t-test; *p<0.05; **p<0.01; ***p<0.001.

FIGS. 5A-5D: Quantitative proteomics to validate the induction of BRD4 O-GlcNAcylation in a site specific manner. (A), sample preparation for the O-GlcNAcylation sites identification by LC-MS/MS. Same samples were submitted to immunoblot to verify the treatment effects; (B), immunoblot analysis of DMSO/640 nM OGTAC-1/640 nM OGTAC-4 treatment effects; Each treatment contains three biological repeats (n=3); (C), Heatmap of BRD4 O-GlcNAcylation sites after treatment by LC-MS/MS. Only HexNAcylation were detected in all three repeats were counted into quantification; (D), Reported PTMs and novel O-GlcNAcylation sites induced by OGTACs in this paper on full length BRD4. NPS, N-terminal phosphorylation site; ET, extra-terminal domain; CPS, C-terminal phosphorylation site; HAT, Histone acetyltransferases; CTM, C-terminal domain.

FIG. 6A: fOGT: HTN-BRD4 plasmid ratio optimisation. Co-transfection of pHTN-BRD4 5 82 g with 0.25 μg (20:1) was selected for later study.

FIG. 6B: Evaluation of HTN-CK2α O-GlcNAc level from whole cell lysate WB. Overlapping the pan-RL2 (red) with HTN-CK2α (green at˜75 kDa) reveals the HTN-CK2αspecific O-GlcNAc level.

FIG. 6C: Evaluation of O-GlcNAc inducing effects of OGTAC-1/2/3 on HTN-CK2α from whole cell lysate WB. OGTAC-1 induced higher fold increase of O-GlcNAcylation on HTN-CK2α both at 500 nM and 1 μM.

FIG. 6D: OGTAC-1 dose-dependent O-GlcNAc inducing effect on HTN-CK2α after 4 h treatment.

FIG. 6E: OGTAC-1 dose-dependent O-GlcNAc inducing effect on HTN-CK2α after 24 h treatment.

FIG. 6F: Immunoblot analysis of OGTAC-1 mediated O-GlcNAcylation when co-treated with 10x concentration of binding competitors.

FIGS. 7A-7E: Specificity and reversibility of OGTAC strategy. (A), Under HTN-CK2α:fOGT=1:0.01 co-transfection system, the cellular global O-GlcNAc level was not significantly changed with or without OGTAC-1 (125 nM) treatment. (B), Constructs of fOGT with 13.5 (rounded to 13) TPR and 0TPR-fOGT (0tfOGT). (C). 0tfOGT but not fOGT did not significantly change the cellular global O-GlcNAc level. (D), Dose-dependent O-GlcNAcylation profile of HTN-BRD4 by OGTAC-4 treatment. HEK293T cells expressing HTN-BRD4:0tfOGT=1:0.05 were treated with an increasing concentration of OGTAC-4 for 4 h. Then, the O-GlcNAcylation level of HTN-BRD4 was assessed by immunoblot after IP using BRD4 antibody. All quantifications are shown as mean±s.e.m. of n=3 biologically independent experiments. (E), Reversible effect of OGTAC-4 on HTN-BRD4 can be achieved by addition of AP1867. HEK293T cells expressing HTN-BRD4:0tfOGT=1:0.05 were pretreated with OGTAC-4 (500 nM) or DMSO for 4 h, and culturing media were replaced by fresh media with DMSO or AP1867 (50 μM) for the indicated time. The quantifications in right panel were conducted by the normalizing the fold-change (OGTAC-4/DMSO) for each post-washout time point to the value of 4 h pre-treatment. All quantifications are shown as mean±s.e.m. of 3 independent biological repeats. Statistical significance was calculated with ordinary one-way ANOVA comparing samples with different transfection conditions or OGTAC-treatment. ns, p≥0.05; *p<0.05; **p<0.01; ***p<0.001; ****p<0.0001.

FIG. 8: Representative immunoblots analysis for CETSA experiments. All probes were treated at 5 μM for 4 h in HEK293T cells expressing HTN-BRD4:fOGT=1:0.05.

FIG. 9: HTN-CK2α:fOGT plasmid ratio optimisation. Co-transfection of 5 μg pHTN-CK2α with 0.05 μg fOGT (1:0.01) (red) was selected for later study.

FIG. 10: Biological repeats of GalT based mass-shift assay to validate the OGTAC-1 induced HTN-CK2α specific O-GlcNAcylation.

FIG. 11: Immunoblotting analysis of time-dependent effects of OGTAC-1 on HTN-CK2α. OGTAC-1H was an inactive, negative control molecule of OGTAC-1. The quantification was conducted by immunoblot signal of total RL2 (except the band specific for HTN-CK2α) relative to GAPDH as the mean±s.e.m. of n=3 biologically independent experiments. Statistical significance was calculated by multiple unpaired t-tests. ns, **p<0.01, ****p<0.0001.

FIG. 12: Immunoblotting analysis of effects of OGTAC-1 on HTN-CK2αin HeLa cell line. HeLa cells were transfected with fOGT:HTN-CK2α=1:0.1 for 24 h, followed by treatment of OGTAC-1 (125 nM) or other probes for 8 h. For OGTAC-1H, ‘++’ denotes 10-fold concentration of OGTAC-1, while “+” denote the same concentration of OGTAC-1. The quantification was conducted by immunoblot signal of RL2 relative to CK2α. as the mean±s.e.m. of n=3 biologically independent experiments. Statistical significance was calculated by unpaired t-tests.

FIG. 13: Evaluation of O-GlcNAc inducing effects of OGTAC-1/2/3 on HTN-EZH2 and dose-dependent effect of OGTAC-1 on HTN-EZH2. In the treatment group, transfection ratio is HTN-EZH2:fOGT:=1:0.01.

FIG. 14: Evaluation of O-GlcNAc inducing effects at wider concentration range of OGTAC-4 on HTN-BRD4 by IP-WB method. The quantification was conducted by immunoblot signal of RL2/HTN-BRD4 as the mean±s.e.m. of n=2 biologically independent experiments. For 3 nM of OGTAC-4, the experiment was conducted once.

FIG. 15: Immunoblotting analysis of OGTAC-4 inducing effect in HTN-BRD4:fOGT=1:0.01 system. No O-GlcNAcylation inducing effect of OGTAC-4 on HTN-BRD4 was observed.

FIG. 16: Immunoblotting analysis of the specificity of our chemogenetic system with HTN-CK2α:fOGT=1:0.01. OGTAC-1 was used at 125 nM.

FIG. 17: Immunoblotting analysis of the O-GlcNAcylation inducing effect of truncated fOGT (tfOGT) on global cellular proteins. The quantification was conducted by immunoblot signal of pan-RL2/GAPDH as the mean±s.e.m. of n32 2 biologically independent experiments. TR, transfection reagent.

FIGS. 18A and 18B: Immunoblotting analysis of the global O-GlcNAcylation inducing effect of truncated OGTAC-4 and OGTAC-1 on HTN-BRD4 and HTN-CK2α respectively. (A), O-GlcNAcylation inducing effect of OGTAC-4 is dependent on the expression of 0tfOGT. (B), OGTAC-1 induced higher O-GlcNAcylation level comparing to the negative control OGTAC-1H. The chlorine-to-hydrogen substitution renders OGTAC-1H unable to recruit 0tfOGT to HTN-CK2α, suggesting that the inducing effect is 0tfOGT-dependent. The quantification was conducted by immunoblot signal of probe treatment/control as the mean±s.e.m. of n=2 biologically independent experiments. TR, transfection reagent.

FIG. 19: Immunoblotting analysis of global O-GlcNAcylation level during reversibility study. The quantification was calculated by immunoblot signal of total RL2 relative to GAPDH as the mean±s.e.m. of n=3 biologically independent experiments. Statistical significance was calculated by multiple unpaired t-tests. ns, not significant.

### DETAILED DESCRIPTION OF THE INVENTION

Protein O-linked β-N-acetylglucosamine modification (O-GlcNAcylation) plays a crucial role in regulating essential cellular processes. Disrupted O-GlcNAcylation homeostasis has been linked to various human diseases, including cancer, diabetes, and neurodegeneration. However, there is no chemical tools for intervention of protein-and site-specific O-GlcNAc modification, rendering the precise study of O-GlcNAcylation formidable. To address this challenge, we have developed first-in-class heterobifunctional small molecules, named O-GlcNAcylation targeting chimeras (OGTACs), which enable protein-specific O-GlcNAcylation in cells. OGTACs promote O-GlcNAcylation of proteins such as BRD4, Ck2α, and p53 in cellulo by recruiting their proximity with FKBP12^{F36V} fused-OGT (fOGT), with temporal and magnitude control. Furthermore. we have identified the O-GlcNAcylation sites of BRD4 after probe treatment. Overall, OGTACs represent a promising approach for inducing protein-specific O-GlcNAcylation for functional dissection and therapeutic applications in the future Such approach can be extended and applied to study other glycan transferases and their glycation sites.

### I. DEFINITIONS

As used herein, the term “proximity inducing protein pair” refers to a pair of proteins in which the first protein can bind to a portion of a proximity inducing ligand and the second protein can bind to another portion of the proximity inducing ligand. The proximity inducing protein pair can be used in methods described herein to label a protein of interest (POI) with a glycan. For example, one protein in the pair can be fused to the glycan transferase and another protein in the pair can be fused to the POI Upon contact and incubation with the proximity inducing ligand, the two proteins in the proximity inducing protein pair bind to the proximity inducing ligand, hence, bringing the glycan transferase and the POI in close proximity to each other for glycan labeling to occur.

As used here, in some embodiments, the term “proximity inducing ligand” refers to a small molecule (e.g., a small organic molecule) having a first portion that can be recognized and bound by one protein of the proximity inducing protein pair and a second portion that can be recognized and bound by the other protein of the proximity inducing protein pair. In other embodiments, the proximity inducing ligand has one portion that can be recognized and bound by the protein fused to the glycan transferase and another portion that can be recognized and bound by the POI. The two portions in the proximity inducing ligand can be joined by a covalent bond or a chemical linker (e.g., a PEG linker or an aliphatic linker (e.g., C2-C8 linker, e.g., C2, C3, C4, C5, C6, C7, or C8 linker)). In some embodiments, the binding is covalent. In other embodiments. the binding is non-covalent.

As used herein, in some embodiments, the term “chase ligand” refers to a small molecule (e.g., a small organic molecule) that can be recognized and bound by the protein (e.g., a protein in the proximity inducing protein pair) that is fused to the POI. In other embodiments, the chase ligand can be recognized and bound by the POI directly. The chase ligand functions as a tool to assess the remaining portion of the POI that is not labeled by the glycan transferase. For example, the protein fused to the POI can be a HaloTag and the chase ligand can be a ligand for HaloTag, e.g., a haloalkane (e.g., a chloroalkane). In some embodiments, the chase ligand also includes a detectable portion, such as a fluorophore, an epitope, or an antibody. As described further herein, in the case that a HaloTag is fused to the POI, once the sample is incubated with the proximity inducing ligand and the glycan transferase has labeled the POI with the glycan, a chase ligand can be added such that any remaining HaloTag-POI fusion protein that is not bound by the proximity inducing ligand can be labeled by the chase ligand.

As used herein, the term “HaloTag” refers to a modified haloalkane dehydrogenase protein that is designed to specifically and covalently bind to haloalkane ligands. Descriptions of HaloTags and their uses are known in the art and can be found in, e.g., Los et al., ACS Chem Biol, 3(6):373-82, 2008. England and Luo, Bioconjug Chem. 26(6):975-86, 2015, and Hoelzel and Zhang, Chembiochem. 21(14):1935-1946, 2020.

As used herein, the term “haloalkane” refers to a small organic molecule that is a ligand for a HaloTag protein. Haloalkanes are also referred to as alkyl halides A haloalkane can include an alkane (e.g., C2-C30 alkane, C2-C20 alkane, C2-C10 alkane, or C2-C5 alkane) and a halogen atom (e.g., F, Cl, Br, or I).

As used herein, the term “linker” refers to a linkage between two elements, e.g., proteins or chemical moieties or portions. A linker can be a covalent bond, a peptide linker, or a chemical linker (e.g., a PEG linker or an aliphatic linker (e.g., C2-C8 linker, e.g., C2, C3, C4, C5, C6, C7, or C8 linker)). The term “bond” refers to a chemical bond, e.g., an amide bond or a disulfide bond, or any kind of bond created from a chemical reaction, e.g., chemical conjugation. The term “chemical linker” refers to a chemical moiety, e.g., a polyethylene glycol (PEG) polymer, that can be used to connect proteins or chemical portions (e.g., chemical portions). The term “peptide linker” refers to an amino acid sequence of a certain length occurring between two polypeptides or proteins to provide space and/or flexibility between the two polypeptides or proteins.

### II. PROXIMITY INDUCING PROTEIN PAIRS AND PROXIMITY INDUCING LIGANDS

As described herein, in some embodiments, the methods utilize a pair of proximity inducing protein pairs, together with a proximity inducing ligand, to bring into proximity the glycan transferase and the protein of interest (POI). In some embodiments, the glycan transferase can be fused with a first proximity inducing protein in the proximity inducing protein pair and the POI can be fused with a second proximity inducing protein in the proximity inducing protein pair. In other embodiments, the glycan transferase can be fused with a second proximity inducing protein in the proximity inducing protein pair and the POI can be fused with a first proximity inducing protein in the proximity inducing protein pair. In certain embodiments, a protein in the proximity inducing protein pair can be any self-labeling protein that has a small molecule ligand. In certain embodiments, a protein in the proximity inducing protein pair can bind to its ligand covalently. In other embodiments, a protein in the proximity inducing protein pair can bind to its ligand non-covalently. Examples of proteins that can be used in a proximity inducing protein pair include, but are not limited to, e.g., FKBP (e.g., FKBP12^{F36V}, with a FKBP specific ligand (e.g., AP1867 ligand)), HaloTag (haloalkane dehalogenase, with haloalkane (e.g., chloroalkane) as a ligand). SNAP-tag (a modified O⁶-alkylguanine-DNA-alkyltransferase, with O⁶-benzylguanine derivatives as ligands), and CLIP-tag (a modified O⁶-alkylguanine-DNA-alkyltransferase, with O⁶-benzylcytosine derivatives as ligands).

Each of the proximity inducing proteins in the protein pair has a small molecule or compound that serves as the ligand to the protein. The proximity inducing ligand is a compound that has one portion that can be bound and recognized by the first proximity inducing protein and another portion that can be bound and recognized by the second proximity inducing protein. In some embodiments, the two portions of the proximity inducing ligand can be connect to each other by way of an optional linker, e.g., a PEG linker. Once the sample containing the protein fusions is in contact with the proximity inducing ligand, the first and second proximity inducing proteins each recognizes and binds to the corresponding portion in the proximity inducing ligand, hence, bringing the first and second proximity inducing proteins, as well as their fusion partners (e.g., glycan transferase and POI), together. As described herein, once the first fusion protein comprising a glycan transferase fused to a first proximity inducing protein and the second fusion protein comprising a POI fused to another proximity inducing protein are brought to close proximity of each other, the glycan transferase can label the POI with a glycan.

Specifically, the glycan transferase used in the methods described herein can be a O-GlcNAc transferase, which can label the POI at the oxygen atom of Ser of Thr residue with the glycan Glc-NAc. In other embodiments, the glycan transferase can be a polypeptide N-Acetylgalactosaminyltransferase 14 (GALNT14) or a glucuronosyltransferase. In certain embodiments of the methods described herein, the glycan transferase can be fused with one protein of the proximity inducing protein pair. e.g., an FK506 binding protein (FKBP) (e.g., FKBP12 with F36V substitution, “FKBP12^{F36V}”). Ligands to FKBP are known in the art. For example, FKBP12^{F36V} can bind to the synthetic ligand AP1867, which can form one portion of the proximity inducing ligand. In some embodiments, the POI can be used with the other protein of the proximity inducing protein pair, e.g., a HaloTag protein, which is a modified haloalkane dehalogenase that can covalently bind to haloalkane ligands (e.g., a chloroalkane). In some embodiments, a haloalkane ligand (e.g., a chloroalkane) can be another portion of the proximity inducing ligand. In some embodiments, a proximity inducing ligand used in methods described herein can contain a first portion that is a ligand to FKBP (e.g., AP1867 as a ligand to FKBP12^{F36V}) and a second portion that is a chloroalkane. In certain embodiments, a ligand to FKBP (e.g., AP1867 as a ligand to FKBP12^{F36V}) and chloroalkane can be joined together via a linker, such as a PEG linker.

Further, a chase ligand can be used in the methods described herein to assess the remaining amount of POI that is not brought to proximity with the glycan transferase. In some embodiments, a chase ligand is a ligand for the protein in the proximity inducing protein pair that is fused to the POI. For example, fusion proteins containing the HaloTag and the POI that are not brought to proximity with the fusion proteins containing the glycan transferase (e.g., O-GlcNAc transferase, polypeptide N-Acetylgalactosaminyltransferase 14 (GALNT14), or glucuronosyltransferase) and the FKBP12^{F36V} can be labeled with a chase ligand. A chase ligand can contain a first portion that is recognized and bound by the protein of the proximity inducing protein pair that is fused to the POI (e.g., HaloTag) and a second portion that provides a detectable readout (e.g., a fluorophore, an epitope that can be detected by an antibody on a Western blot). In some embodiments, the first portion and the second portion in a chase ligand can be joined to each other via an optional linker. In some embodiments, the second portion is an optionally substituted BET bromodomain inhibitor (JQ1), an optionally substituted EZH2 inhibitor, an optionally substituted BRD inhibitor, or an optionally substituted kinase inhibitor. In some embodiments, the second portion is an optionally substituted BET bromodomain inhibitor (JQ1).

Further, in some embodiments, the disclosure also provides methods for labeling a protein of interest (POI) with a glycan, comprising: (a) contacting, in a sample, a fusion protein comprising a glycan transferase fused to a proximity inducing protein, and the POI, with a proximity inducing ligand; and (b) incubating the sample for a duration sufficient for the fusion protein and the POI to both to bind to the proximity inducing ligand and for the glycan transferase to label the POI with a glycan. In this case, the POI is not fused to another protein. In some embodiments, the proximity inducing ligand comprises one portion that can be recognized and bound by the proximity inducing protein and another portion that can be recognized and bound by the POI. While the sample is incubated with the proximity inducing ligand, the fusion protein comprising the glycan transferase and the proximity inducing protein and the POI are brought into close proximity to each other for the glycan transferase to label the POI with a glycan.

Moreover, the amount of POI that remains unlabeled by the glycan transferase can be assessed and measured by incubating the sample with a chase ligand that can be directly recognized and bound by the POI. In certain embodiments, the chase ligand comprises a detectable portion (e.g., a fluorophore) and a portion that is a ligand for the POI.

### III. LINKER

In some embodiments, an optional linker can be used between the first or second proximity inducing protein and the glycan transferase. In some embodiments, an optional linker can be used between the first or second proximity inducing protein and the POI. A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein can react with the amino group at the N-terminus of another protein in a condensation reaction to form a peptide bond. Specifically, the peptide bond can be formed from synthetic means through a conventional organic chemistry reaction well-known in the art, or by natural production from a host cell, wherein a nucleic acid molecule encoding the DNA sequences of both proteins, e.g., a glycan transferase and a first or second proximity inducing protein of the proximity inducing protein pair, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, an aliphatic linker (e.g., C2-C8), the polymer can be functionalized with reactive chemical functional groups at each end to react with the terminal amino acids at the connecting ends of two proteins. In certain embodiments, a synthetic polymer, e.g., a PEG polymer, can be used in the proximity inducing ligand. In certain embodiments, a synthetic polymer, e.g., a PEG polymer, can be used in the proximity inducing ligand between the portion that can be recognized and bound by the first protein in the proximity inducing protein pair and the portion that can be recognized and bound by the second protein in the proximity inducing protein pair. In certain embodiments, a synthetic polymer, e.g., a PEG polymer, can be used in the proximity inducing ligand between the portion that can be recognized and bound by the proximity inducing protein and the portion that can be recognized and bound by the POI.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react through synthetic chemistry means to form a chemical bond, thus connecting the two proteins together. Such chemical conjugation procedures are routine for those skilled in the art.

Suitable peptide linkers that are known in the art include, for example, peptide linkers containing flexible amino acid residues such as glycine and serine. In certain embodiments, a linker can contain peptide linker motifs, e.g., multiple or repeating motifs, of GS, GGS, GGG (SEQ ID NO:1). GGGGS (SEQ ID NO:2), GGSG (SEQ ID NO:3), or SGGG (SEQ ID NO:4). In certain embodiments, a linker can contain 2 to 12 amino acids including motifs of GS, e.g., GS, GSGS (SEQ ID NO:13), GSGSGS (SEQ ID NO:14), GSGSGSGS (SEQ ID NO:15), GSGSGSGSGS (SEQ ID NO:5), or GSGSGSGSGSGS (SEQ ID NO:6). In certain other embodiments, a linker can contain 3 to 12 amino acids including motifs of GGS. e.g., GGS, GGSGGS (SEQ ID NO:7), GGSGGSGGS (SEQ ID NO:8), and GGSGGSGGSGGS (SEQ ID NO:9). In yet other embodiments, a linker can contain 4 to 12 amino acids including motifs of GGSG (SEQ ID NO:3), e.g., GGSG (SEQ ID NO:3), GGSGGGSG (SEQ ID NO:10), or GGSGGGSGGGSG (SEQ ID NO:11). In other embodiments, a linker can contain motifs of GGGGS (SEQ ID NO:2), e.g., GGGGSGGGGSGGGGS (SEQ ID NO:12). In other embodiments, a linker can also contain amino acids other than glycine and serine. The length of the peptide linker and the amino acids used can be adjusted depending on the two proteins involved and the degree of flexibility desired in the final protein fusion. The length of the linker can be adjusted to ensure proper protein folding and avoid aggregate formation. It is understood that the linker length will depend on the context that it is used, and that one of ordinary skill in the art will be able to ascertain the adequate length using standard techniques.

### IV. CHASE LIGANDS

As described herein, a fusion protein containing a glycan transferase and one protein of the proximity inducing protein pair can be used to label a POI fused to another protein of the proximity inducing protein pair with a glycan by incubating a sample containing the two fusion proteins with a proximity inducing ligand. A chase ligand can be used as a tool to assess the remaining portion of the POI that is not labeled by the glycan transferase. In some embodiments, the chase ligand can contain two portions: a first portion that can be recognized and bound by the protein of the proximity inducing protein pair that is fused to the POI and a second portion that is a detectable moiety. In other embodiments, the chase ligand can contain two portions: a first portion that can be directly recognized and bound by the POI and a second portion that is a detectable moiety. A detectable moiety or portion serves as a tool to allow the measurement and quantification of the amount of POI that isn't labeled with the glycan by the glycan transferase. Examples of a detectable moiety or portion include, but are not limited to, a fluorophore, an epitope, or an antibody.

For example, the protein fused to the POI can be a HaloTag and the chase ligand can be a ligand for HaloTag. e.g., a haloalkane (e.g., a chloroalkane). As described further herein, in the case that a HaloTag is fused to the POI, once the sample is incubated with the proximity inducing ligand and the glycan transferase has labeled the POI with the glycan, a chase ligand can be added such that any remaining HaloTag-POI fusion protein that is not bound by the proximity inducing ligand can be labeled by the chase ligand.

### V. EXAMPLE

### Introduction

Protein O-linked β-N-acetylglucosamine modification (O-GlcNAcylation) plays a significant role in the regulation of transcription, metabolism, cell signalling, protein stability, and nucleocytoplasmic trafficking.¹⁻⁴ Increasing amount of research uncovered that abnormal regulation of O-GlcNAcylation related to many pathological disorders, including cancer,⁴⁻⁸ neurodegenerative diseases,⁹ cardiovascular diseases,¹⁰ autoimmune diseases,¹¹ and diabetes.¹²

However, the functional roles of O-GlcNAcylation are believed to be protein-specific, and the dissections of their functions are challenging and incomplete. Current strategy used to study protein-specific functions are either highly genetic engineering dependent, which includes glycosite genetic mutation,^{13,14} and nanobody-induced OGT-substrate proximity in cellulo;^{15,16} or in vitro based, for example semi-synthesis by peptide-protein ligation.¹⁷ Although successfully illustrated the specific functions of their proteins of interest (POIs), these methods all necessitate extensive cell engineering and not generalized to be used as therapeutic interventions.

In contrast, chemical inhibitors of O-GlcNAc transferase (OGT) and O-GlcNAcase (OGA), the pair of enzymes that catalyzing the addition and removal of O-GlcNAcylation are intensively studied due to their therapeutic potential.¹⁸ However, as OGT and OGA are the only pair of enzymes regulating the O-GlcNAcylation of over thousands of nuclear and cytosolic proteins, the inhibition of inhibiting these enzymes will inevitably disrupt the global cellular O-GlcNAcylation homeostasis, rendering the complicated, off-targeted downstream effects. Moreover, prolonged treatment with these inhibitors could trigger feedback regulation of OGT/OGA, leading to potential drug resistance.^{19,20}

Encouraged by recent development of chemical inducer of phosphorylation^{21,22} and acetylation,²³ we hypothesize a heterobifunctional system which recruits OGT to the target protein can achieve target specific O-GlcNAcylation.

Here, we present the development of O-GlcNAcylation targeting chimeras (OGTACs) which induce O-GlcNAcylation of POIs, including BRD4, Ck2α and EZH2,through promoting their proximity with FKBP12^{F36V} fused-OGT (fOGT). We optimise the molecular structure of OGTACs, and validate that their O-GlcNAcylation inducing effect is dose, time dependent. Furthermore, through protein LC-MS/MS, we demonstrated that OGTACs induced site-specific O-GlcNAcylation on BRD4.

### Results

### Design of OGTACs to Induce General Halotag-Fused Protein O-GlcNAcylation in Cells

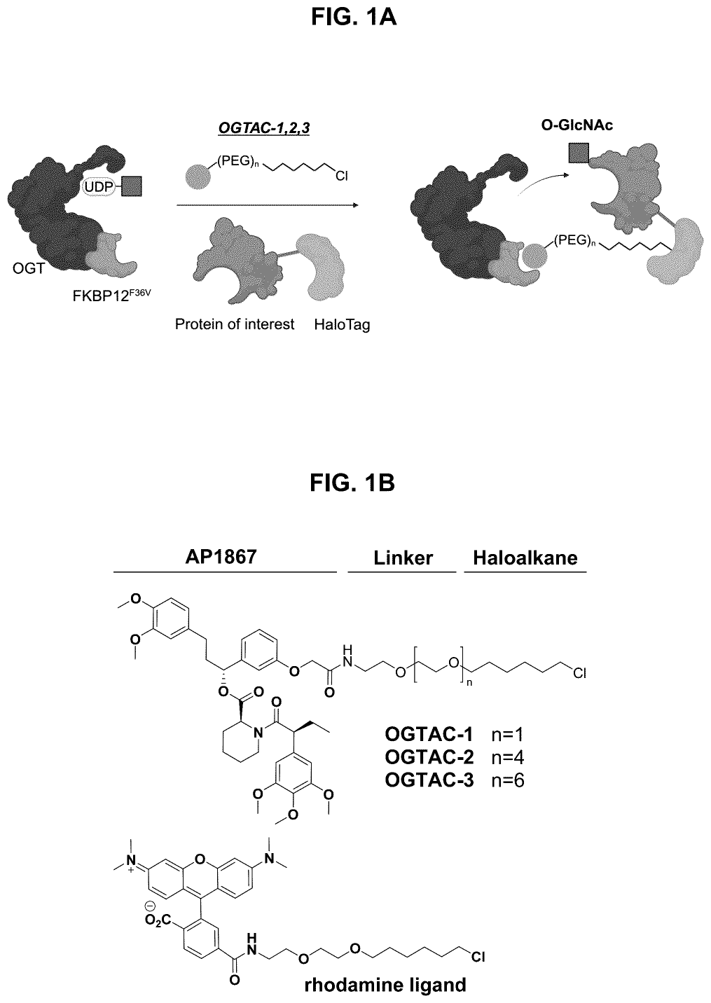

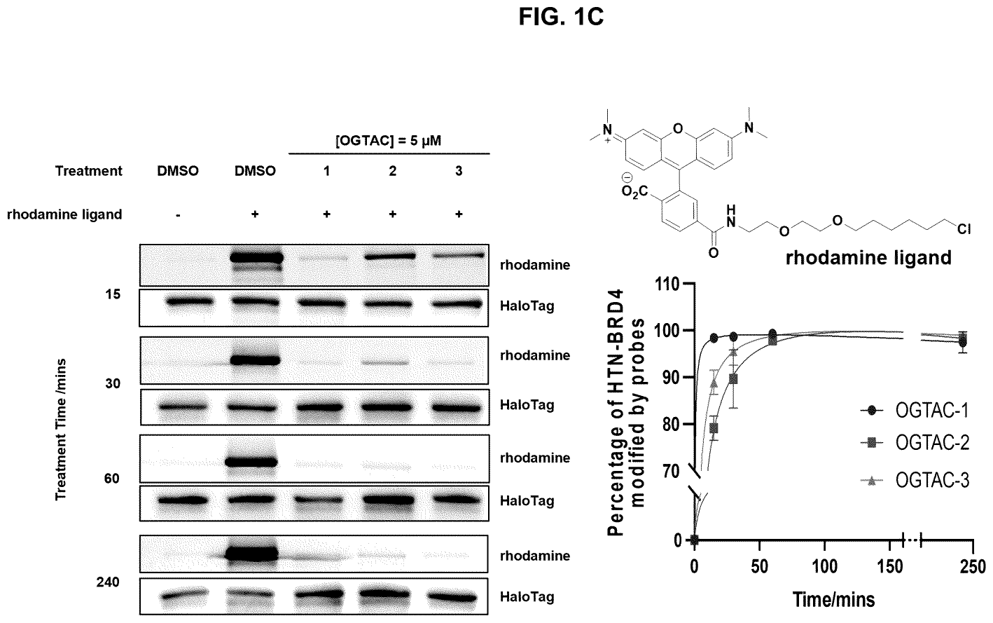

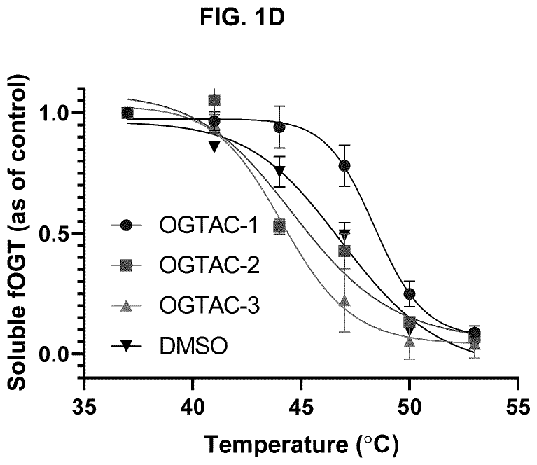

To demonstrate the feasibility of induction of protein specific O-GlcNAcylation by chemical induced proximity (CIP), we first established a general, well-studied tagged system for proof-of-concept study. Since there is currently no non-inhibitory ligand available for OGT, we expressed FKBP12^{F36V} fused-OGT, which can be efficiently recruited by AP1867 motif. Halotag-fused POIs were recombinantly expressed as our O-GlcNAcylation target, which can be covalently linked by haloalkane. Therefore, we synthesize a series of molecules linking AP1867 and haloalkane through different repeats of polyethylene glycol (PEG), namely O-GlcNAcylation-Targeting Chimera (OGTAC) to induce proximity between fOGT and Halotag-fused POI (FIGS. 1a,b). We optimised the plasmid ratio between FKBP12^{F36V}-OGT and Halotag-fused POI so that we could detect POI's O-GlcNAcylation through immunoblot, while keeping the basal level of O-GlcNAcylation to a minimum, and allowing for a clear display of the O-GlcNAcylation inducing effects of OGTACs.

### OGTAC 1 Induces HTN-BRD4 O-GlcNAcylation in a Dose and Time-Dependent Manner

We selected bromodomain-containing protein 4 (BRD4) as our first POI. BRD4 is an epigenetic regulator that play significant roles in cancer. PTMs, such as phosphorylation, methylation and ubiquitination were well studied; especially phosphorylation was reported to affect its chromatin targeting, onco-factors recruitment, and cancer progression.²⁴ However, O-GlcNAcylation on BRD4 has never being comprehensively studied, mostly due to the lack of appropriate tools.

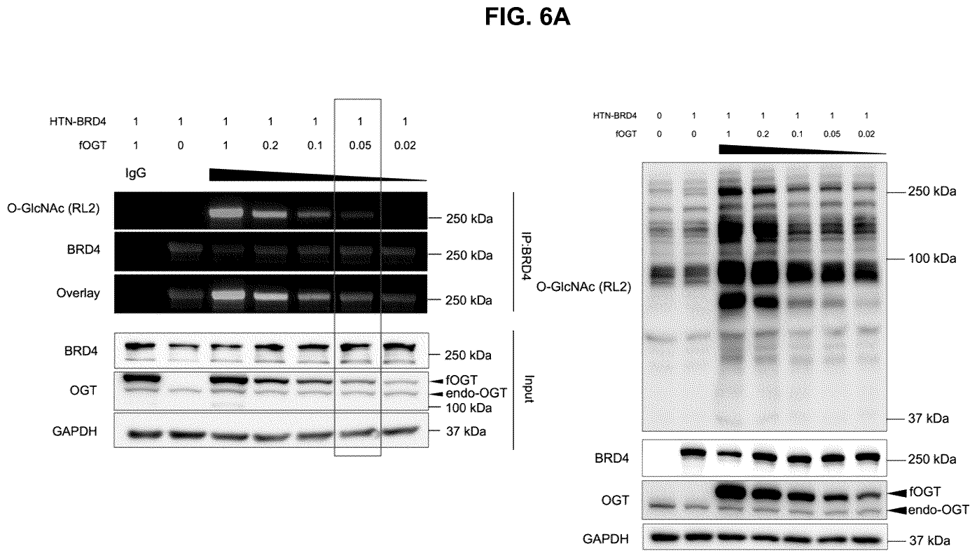

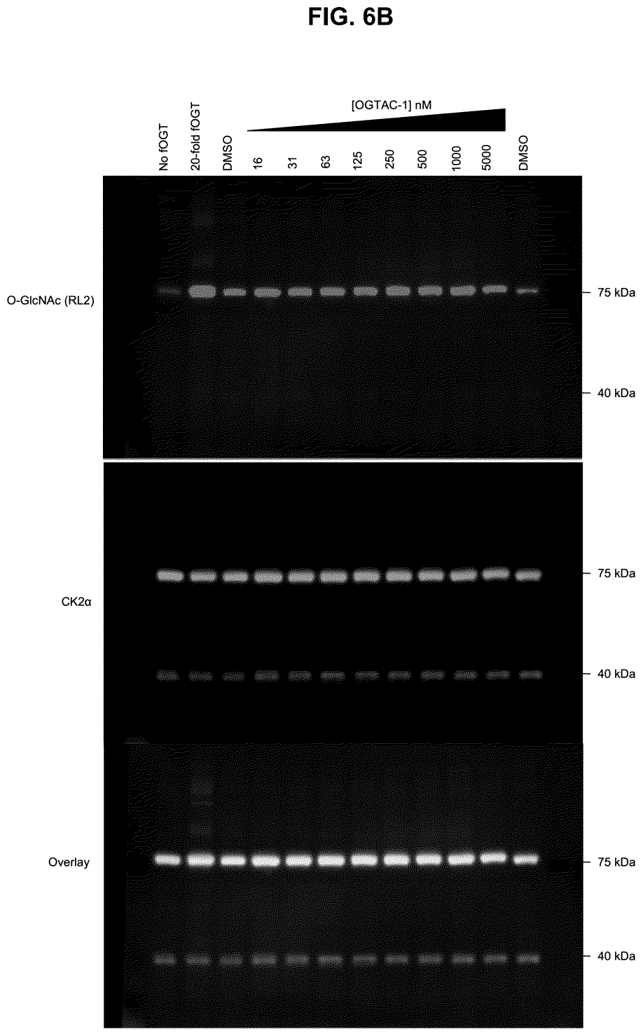

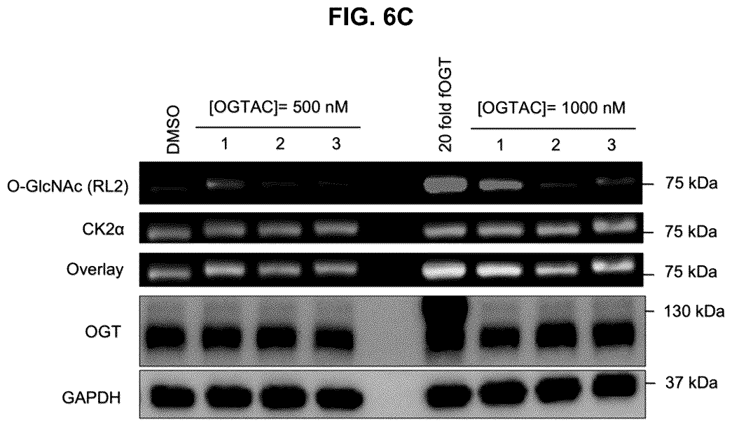

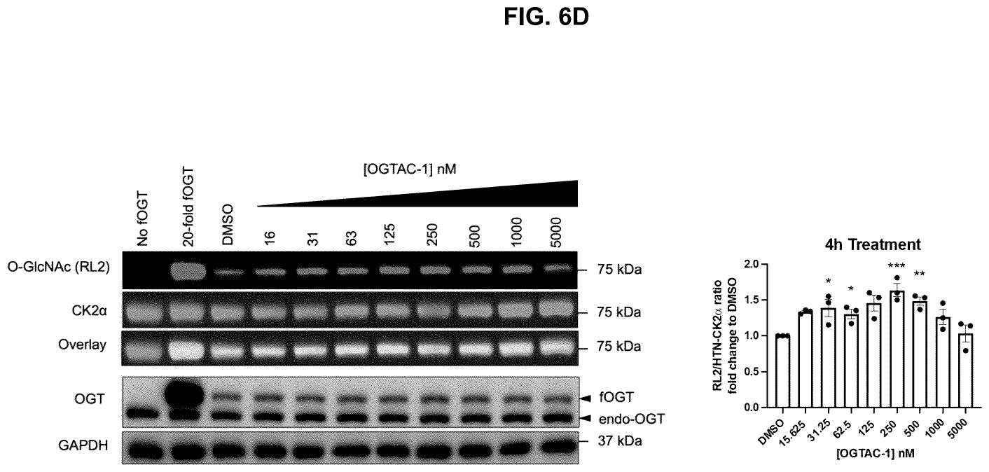

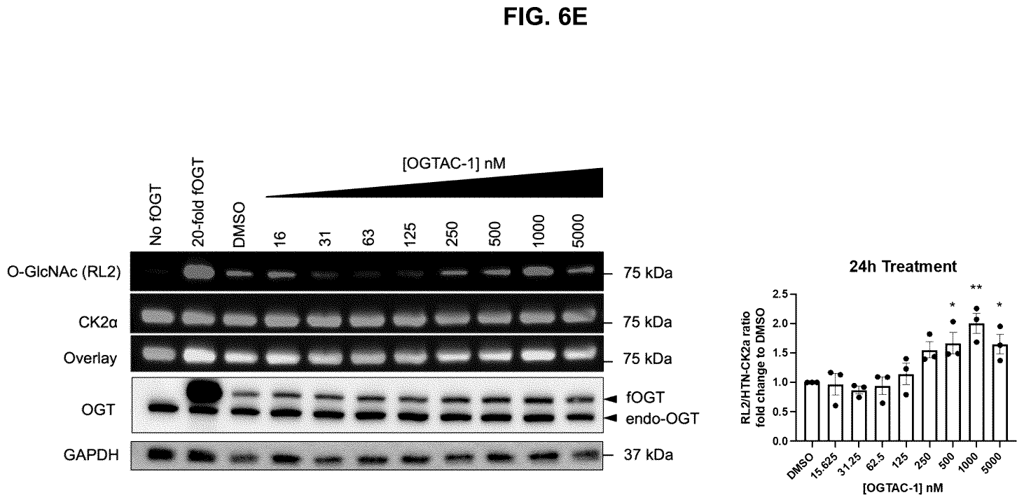

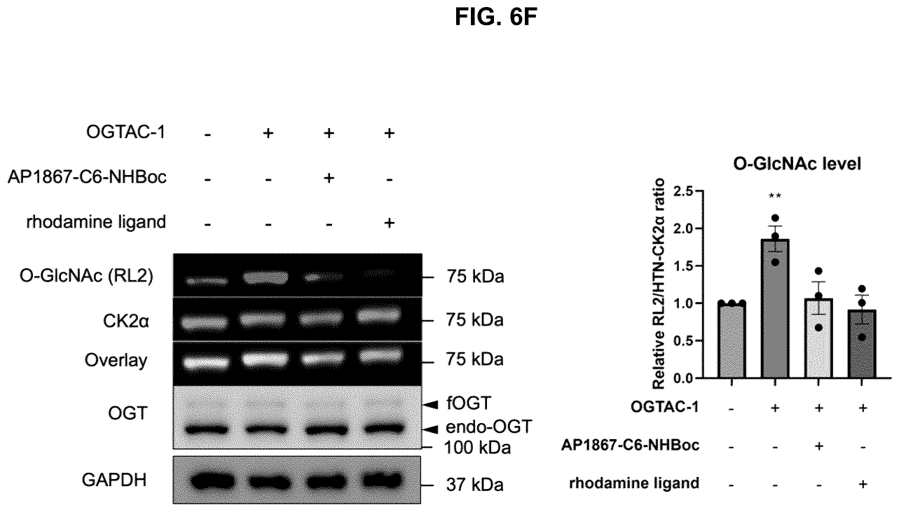

We first constructed the Halotag-C-terminal BRD4 (HTN-BRD4) plasmid and co-transfected it with fOGT plasmids in HEK293T cells at different ratio from 1:1 to 50:1. By immunoprecipitation (IP) and probing for O-GlcNAc using RL2 antibody, we noted that the O-GlcNAc level of HTN-BRD4 is proportional to the amount of fOGT expressed (FIG. 6A). This result indicated that HTN-BRD4 is a neo-substrate of fOGT, and they only started to be O-GlcNAcylated when fOGT expression reached a threshold value (FIG. 6A). We set the co-transfection ratio at 20:1, the threshold that showed minimal O-GlcNAcylation, for our OGTAC induction study.

Before evaluating the inducing effects of OGTACs, we first developed two cell-based assays to verify they can engage HTN-BRD4 and fOGT in cells.

For HTN-BRD4, as the OGTAC-1 to 3 were designed to be irreversibly linked to it, we developed a pulse-chase assay. In this assay, we first treated the HEK293T cells that co-expressing HTN-BRD4 and fOGT to different time of OGTACs; then, we replace the culture media and exposed the cells to TMR-Halo ligand, which fluorescently labelled the HTN-BRD4 that did not interact with OGTACs (FIG. 1C). We set 15 mins, 30 mins, 1 h and 4 h, four treatment time and evaluated engagement potency of 5 μM OGTAC-1 to 3 (FIG. 1C). All of three OGTACs efficiently reached HTN-BRD4, fully occupied the protein within 4 h. OGTAC 1 ranked first among three OGTACs, achieving 22 90% engagement within 15 mins treatment.

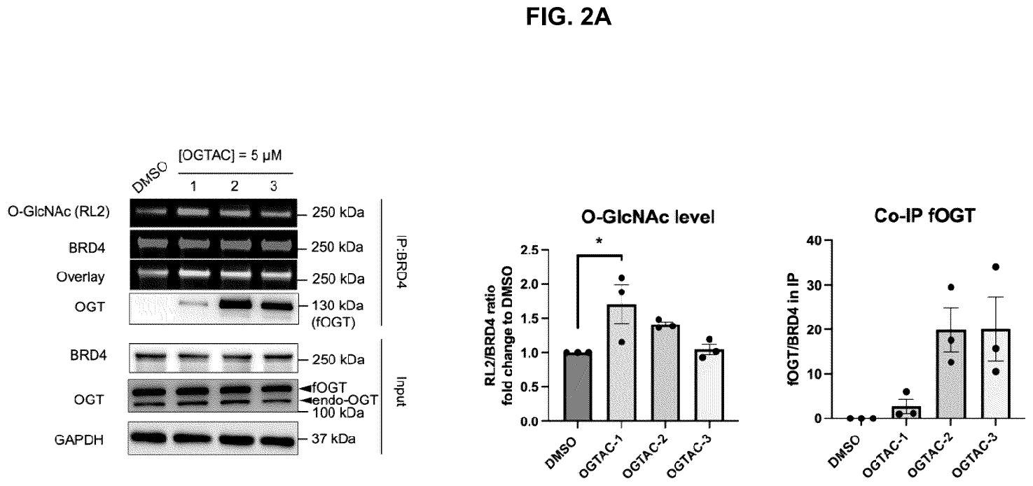

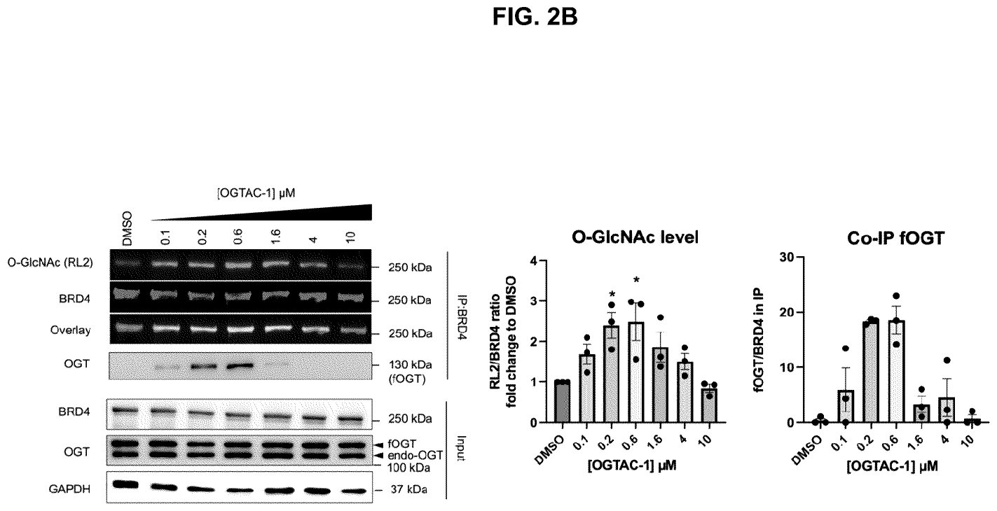

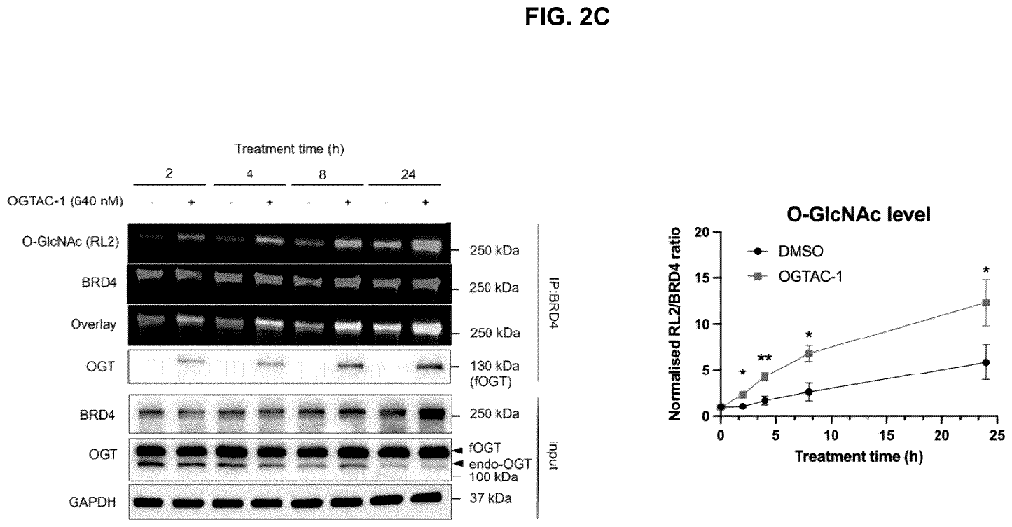

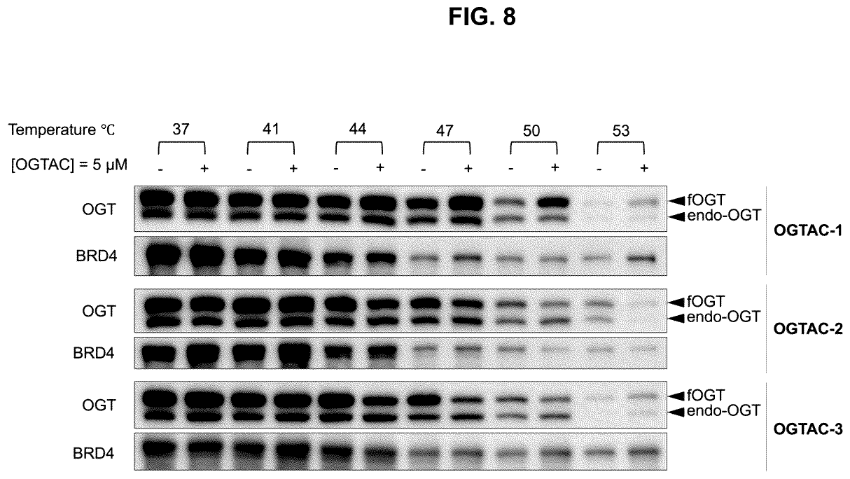

For fOGT engagement, we established a cellular thermal shift assay (CETSA). Using same co-expressing system, we treated cells with OGTAC 1-3 for 4 h, and conducted heat challenge to intact cells at a series of temperature to determine the corresponding melting temperature (Tₘ). All OGTAC treated groups showed increased Tₘ comparing with DMSO control which indicates there are direct binding between OGTACs to fOGT (FIG. 1D, FIG. 8). OGTAC 1 ranked first among three molecules with the highest ΔTₘ (1.4° C.), probably due to its shortest linker length and smaller molecular weight. As the principal of CETSA is to quantified soluble protein after heat shock, we suspect the OGTAC-2 and 3 did not increase Tₘ due to the stable ternary complex they induced (shown next section FIG. 2A). The large ternary complex (molecular weight˜400 kDa) is more likely to become insoluble upon heat challenge.

We next assess their potency in inducing HTN-BRD4 O-GlcNAcylation in cell. Although we identify OGTAC-1 as the most efficient structure in pulse-chase and CETSA, we still cannot ensure it is the most potent structure in terms of inducing HTN-BRD4 O-GlcNAcylation. Therefore, after treating cells with 5 μM OGTAC-1 to 3 for 4 h. we immunoprecipitated BRD4 proteins from cell lysate and measured their O-GlcNAcylation level variation by RL2 antibody. All three OGTACs induced O-GlcNAcylation on HTN-BRD4 but consistent with binding assay. OGTAC-1 showed outstanding potency (FIG. 2A).

We further investigated he dose-dependent effect of OGTAC-1. The inducing effect was bell-shaped, with highest level of O-GlcNAcylation between 256 nM and 640 nM. Co-IP of the fOGT was also observed at these concentrations, which the induction of HTN-BRD4 O-GlcNAcylation is dependent on a stable ternary complex formation (FIG. 2B). The O-GlcNAcylation inducing effect of OGTAC 1 declined from 1.6 μM, demonstrating the hook effect started to occur at this concentration. Due to the low expression level of endogenous BRD4, we could not compare the OGTAC-1 effect on it using immunoblot, but we noted pan-RL2 level was not affected, which supported the inductive effect is target specific.

Following identification of the optimised concentration of OGTAC-1, we evaluated the kinetics of OGTACs-mediated O-GlcNAcylation to reveal its time-dependent effect. We examined the O-GlcNAcylation level change at 2 h, 4 h, 8 h and 24 h. The RL2 signal of BRD4 displayed a 2.5-fold increase at 2 h and further escalated to around 2.8-fold at 4 h (FIG. 2C). However, the longer time seems did not further enhance the inducing effects, possibly due to the limitation of this transient transfection system: although media with transfection reagents and plasmids were removed before OGTAC treatment, the cells are still expressing these recombinant HTN-BRD4 and fOGT. The basal level on BRD4 caused by fOGT was increased after 24 h, and the low concentration (640 nM) may become not optimised for inducing the excessive expressed HTN-BRD4 O-GlcNAcylation.

Collectively, these data suggested that OGTAC induced HTN-BRD4 O-GlcNAcylation in a dose-dependent and time-dependent manner.

### OGTAC Strategy Extended to HTN-Ck2α

Next, to illustrate the generality of OGTAC strategy, we assessed OGTAC-1 to 3 effects on HTN-Casein kinase (HTN-Ck2α).

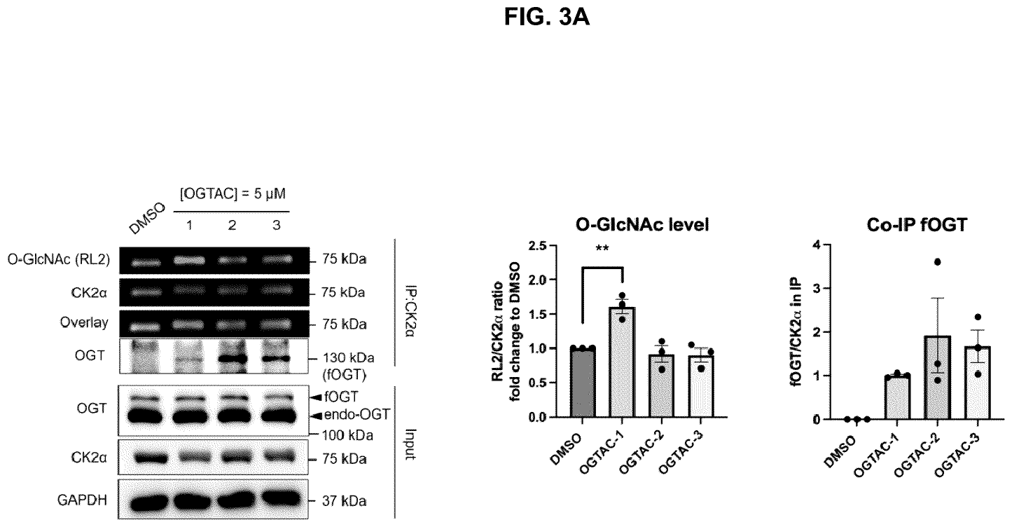

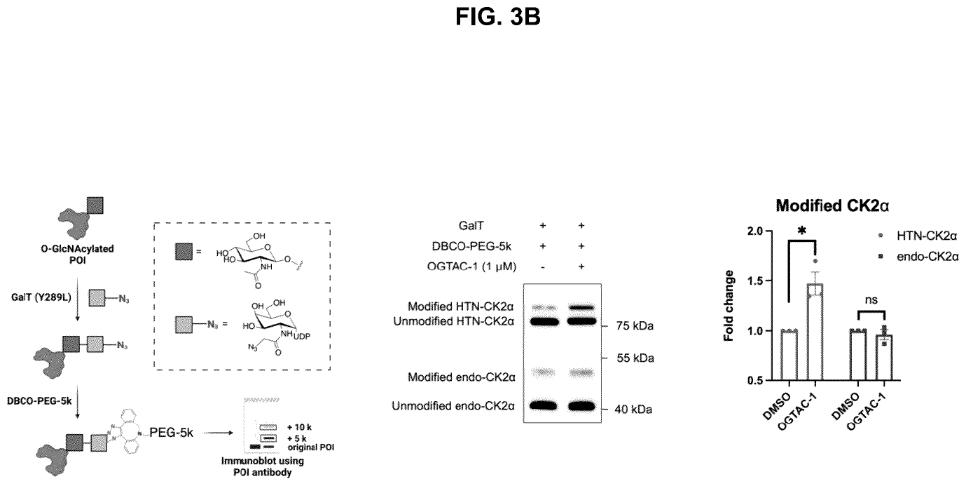

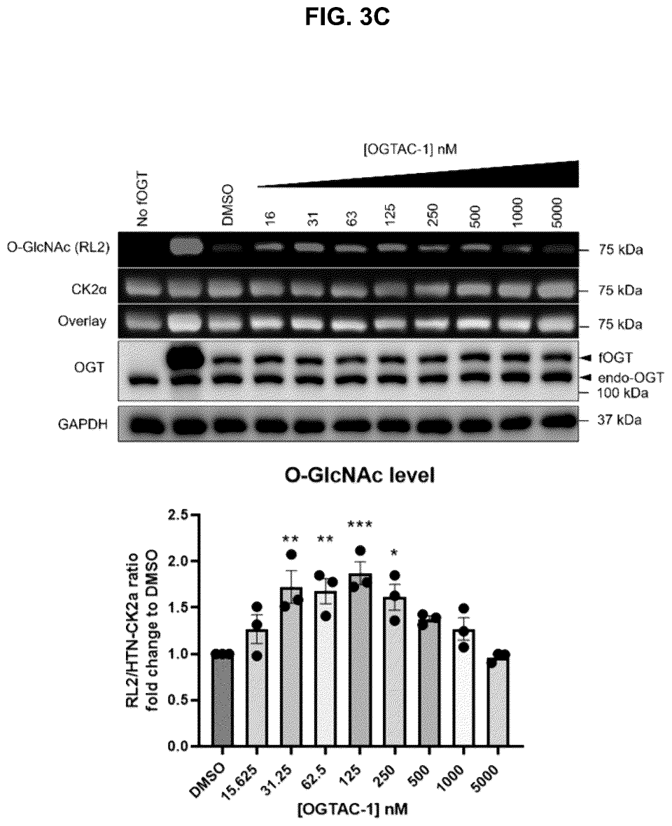

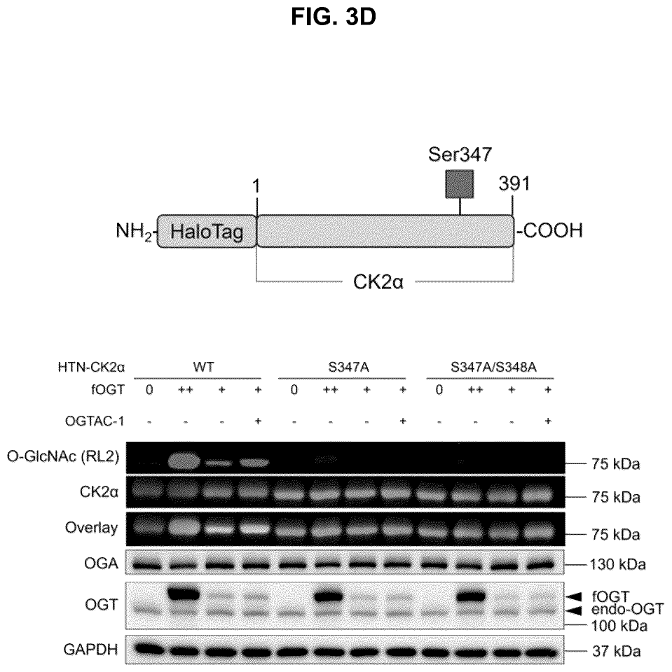

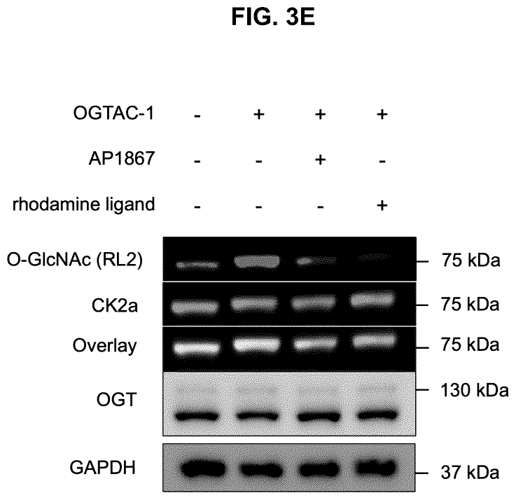

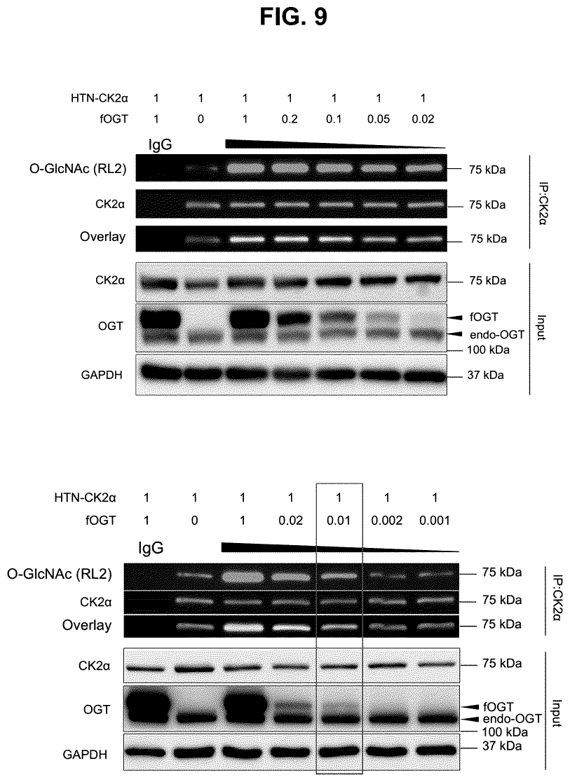

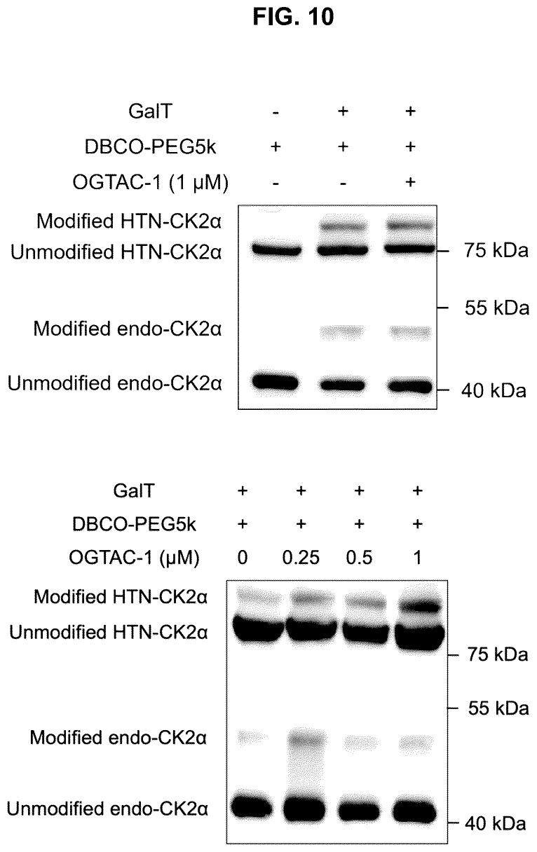

Ck2α is a well-studied OGT substrate.^{17,25} Its Ser347 is conservatively O-GlcNAcylated and this modification was reported to affect its kinase activity.^{16,17} We first co-expressed HTN-Ck2α and fOGT in HEK293T cells at different ratio and found the basal O-GlcNAcylation level of HTN-Ck2α was higher than what we detected in the case of HTN-BRD4. Therefore, a lower ratio of fOGT expression (1:100) was selected for the assessment of OGTAC potency (FIG. 9). OGTAC-1 still prevailed among 3 OGTACs by IP followed by immunoblot (FIG. 3A). The induction of specific O-GlcNAcylation on HTN-Ck2α was further confirmed by mass-shift assay. In this assay, O-GlcNAcylated protein in cell lysate were enzymatically labelled N-azidoacetylgalactosamine (GalNAz), followed by covalent linkage to polyethylene glycol (PEG) of 5 kDa by strain-promoted alkyne-azide cycloaddition (SPAAC), resulting an up-shifted band in immunoblot (FIG. 3B, FIG. 10).²⁶ We validated that 1 μM of OGTAC-1 increased HTN-CK2α O-GlcNAcylation; importantly, OGTAC-1 treatment did not alter the O-GlcNAcylation level of endogenous CK2α, suggesting its target specificity (FIG. 3B, FIG. 10). To validate the direct binding to both fOGT and HTN-Ck2α is critical, we exploited AP 1867 and rhodamine ligand, which bind to fOGT and HTN-Ck2α respectively, to co-treat with OGTAC-1. As we expected, co-treatment with either competitor abolished the O-GlcNAcylation inducing effects if OGTAC-1 (FIG. 3E, FIG. 6F).

During immunoblotting study, we noticed that even for whole cell lysate (WCL), RL2 antibody band (green) and HTN-CK2α protein band (red) are perfectly overlapped and clearly distinguishable from other bands on blot (FIG. 6B). We assessed the OGTAC treatment effects by quantifying the RL2 signal that is overlapping with HTN-Ck2α in WCL, and found they are corresponding with the results achieved by IP-WB method (FIG. 6C). Therefore, we quantify the intensity of RL2 band that overlapping with HTN-CK2α level instead of IP CK2α in later study.

Next, we investigated the dose- and time-dependent effects of OGTAC-1. We observed that, at 250 nM concentration, induction of O-GlcNAcylation happened within 4 h and peaked at 8 h and started to decline (FIG. 3C, FIG. 6D) However, for 24 h treatment, the induction peaked at a higher concentration of OGTAC-1 (1 μM) instead (FIG. 6E). This might be due to the need for a larger population of OGTAC-1 molecules to maintain the appropriate stoichiometry with the increased expression of HTN-CK2α protein. This phenomenon again highlights the importance of optimizing the fOGT:OGTAC POI ratios for efficient chemically induced O-GlcNAcylation.

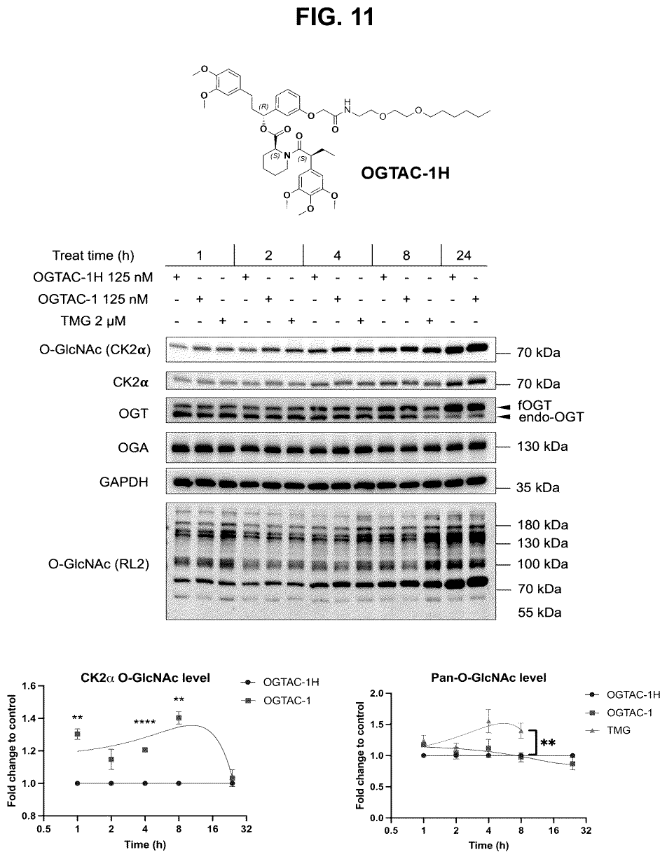

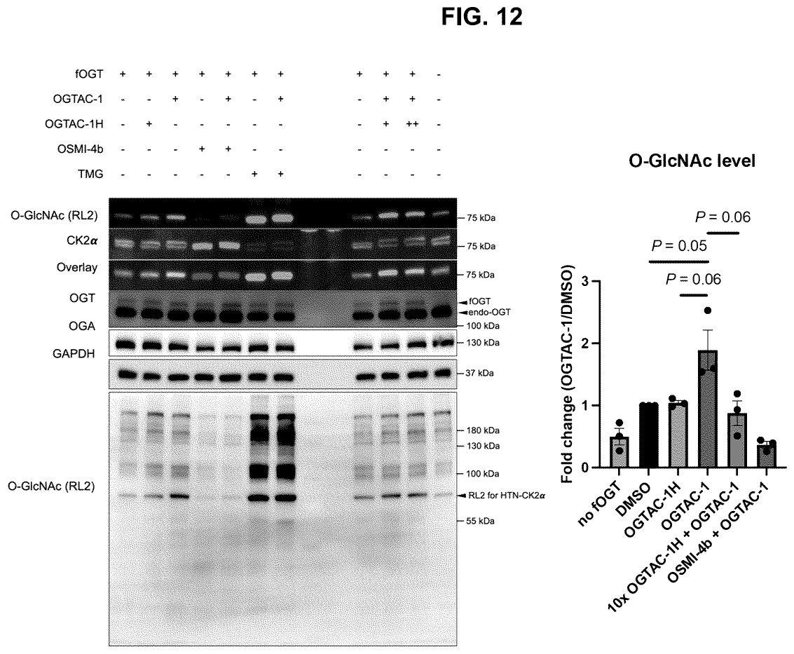

S347 is a well-defined O-GlcNAcylation site on CK2α, while S348 is the potential alternative site as it is adjacent to S347. To verify the O-GlcNAc sites induced by OGTAC-1 in HTN-CK2α. we constructed HTN-CK2α plasmids with S347A and S347A/S348A mutations. Immunoblotting with the RL2 antibody indicates that S347 is a potential O-GlcNAc site on HTN-CK2α, with OGTAC-1 inducing O-GlcNAcylation at this site (FIG. 3D). FIG. 11 further shows immunoblotting analysis of time-dependent effects of OGTAC-1 on HTN-CK2α. However, we must acknowledge the possibility of additional O-GlcNAc sites, given the known motif preferences and biases of RL2 antibody.42,43 Furthermore, the application of the OGTAC strategy in HeLa cells has also confirmed the effect of OGTAC-1 on HTN-CK2α (FIG. 12), thereby ruling out the possibility that its action is limited to a single cell line. Collectively, these findings support the physiological relevance of O-GlcNAcylation induced by OGTAC-1.

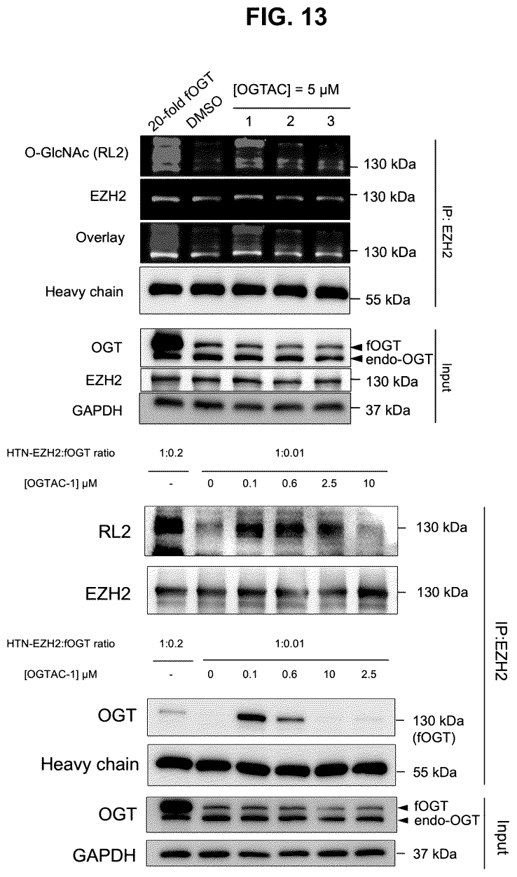

The same strategy was also extended to enhancer of zeste homolog 2 (EZH2), an epigenetic regulator that is an OGT substrate.44,45 OGTAC-1 also demonstrated the highest O-GlcNAcylation-inducing effects among the three OGTACs (FIG. 13). As there are >20,000 commercially available plasmids for HaloTag-fused human proteins,46 OGTACs can be rapidly tested in a wide range of POIs, enabling time- and dose-dependent manipulation of their specific O-GlcNAcylation level.

### Development of OGTACs to Induce BRD4 O-GlcNAcylation Independent of Halotag

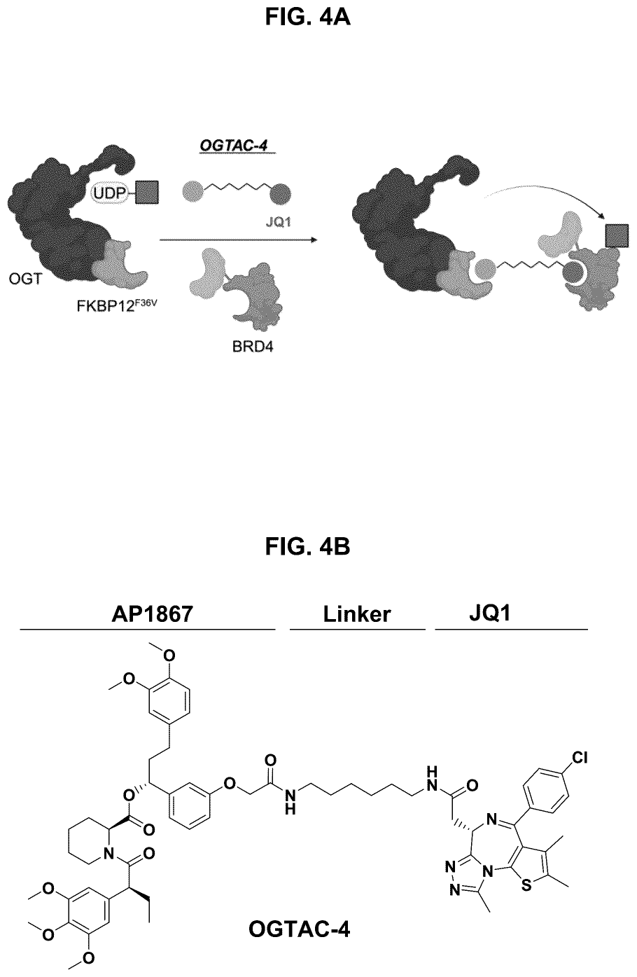

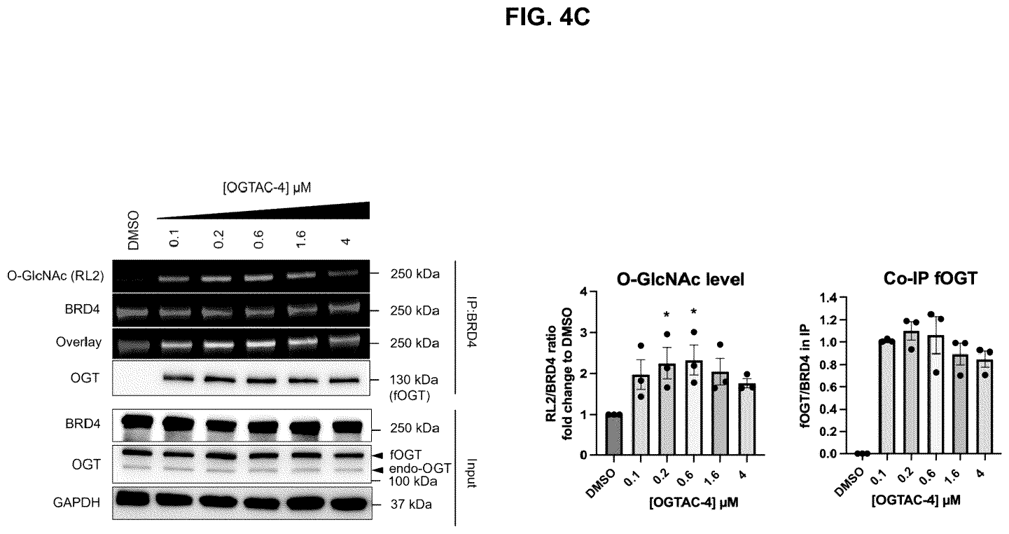

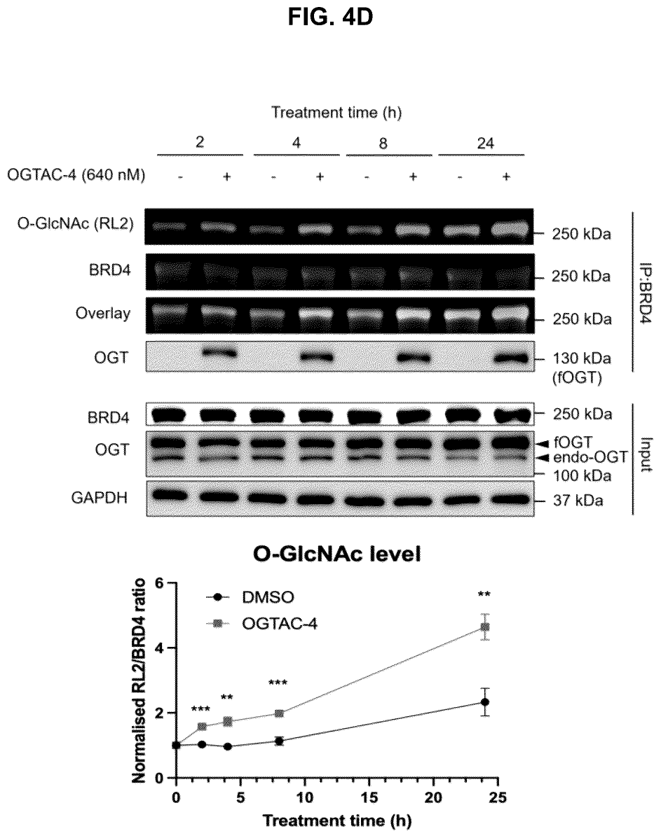

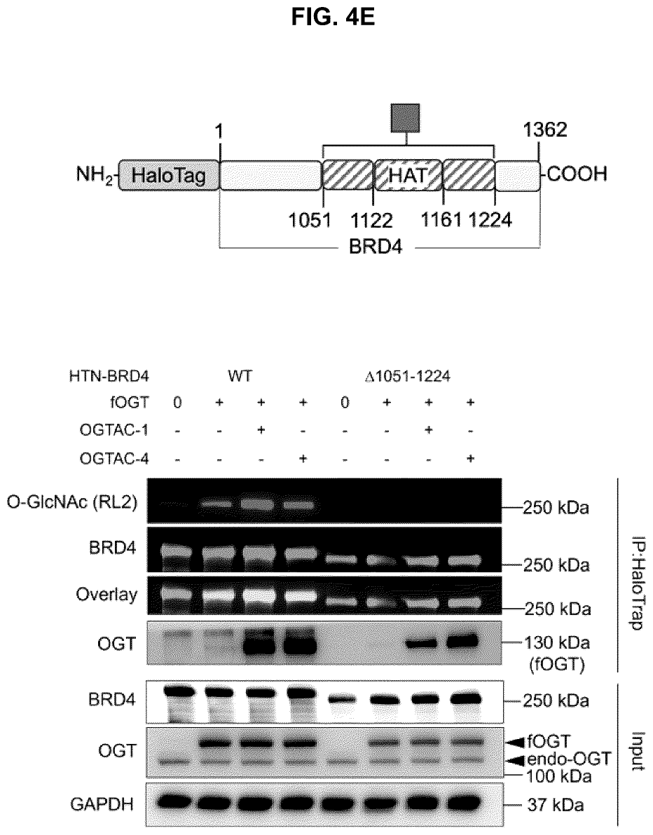

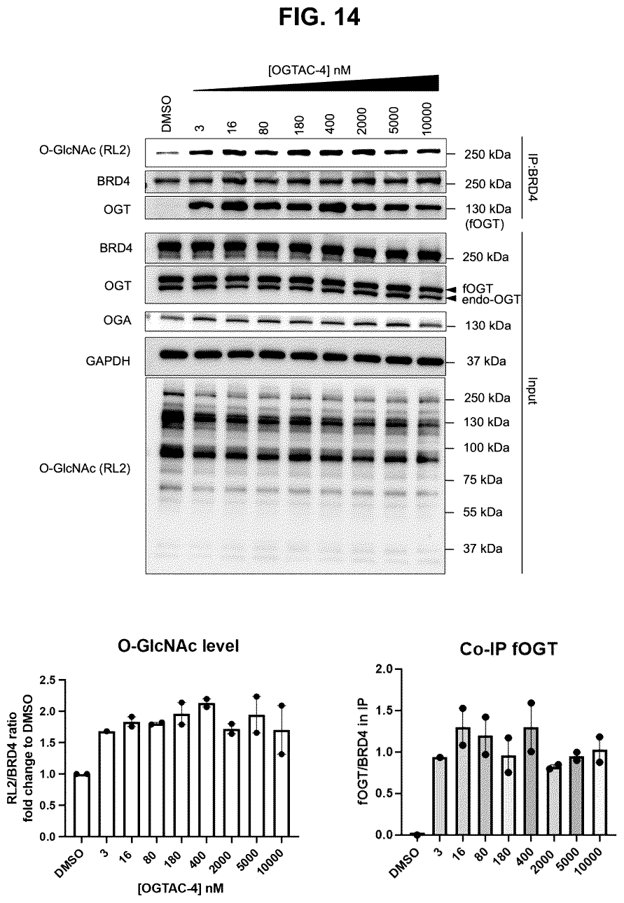

The successful application of OGTAC-1 demonstrated the viability of chemical induced O-GlcNAcylation in a dual fusion-protein system. We next pursued the induction of POI O-GlcNAcylation independent of Halotag; this could facilitate the induction of O-GlcNAcylation on endogenous POIs for their functional dissection. To this end, we synthesized the OGTAC-4, in which a JQ1 structure was incorporated as binding motif for BRD4 BD1/BD2 domain (FIGS. 4A and 4B). As OGTAC-4 directly recruit BRD4, we first try to assess its potency on endogenous BRD4 in cell line stably expressing fOGT. However, we failed to detect any signal from IP-WB method using RL2 as O-GlcNAc detecting antibody. Then, we overexpressed BRD4 together with fOGT in HEK293T cells to accommodate the readout to fit in IP-WB detection limit. As expected, in the co-expression system, we validated the O-GlcNAcylation inducing effect of OGTAC-4. OGTAC-4 also promoted the formation of stable ternary complex between BRD4 and fOGT at range 100 nM to 4 μM (FIG. 4C). Of note, OGTAC-4 promoted BRD4 O-GlcNAcylation and the formation of a stable ternary complex between BRD4 and fOGT at concentrations as low as 3 nM (FIG. 4C & FIG. 14). Comparing with the covalent OGTAC-1, OGTAC-4 demonstrated a wider effective concentration range. This may be due to the sub-stoichiometric catalytic activity of OGTAC-4; at lower concentration, OGTAC-4 can still be effective because after enzymatic transfer of O-GlcNAc from fOGT active site to BRD4, the BRD4 would dissociate from the original ternary complex and other unmodified BRD4 could be recruited to fOGT by OGTAC-4. However, OGTAC-1 covalently linked to HTN-BRD4, and AP1867 binds tightly to fOGT. therefore, once HTN-BRD4 dissociated, free OGTAC-1 molecules may form binary complex with fOGT and inhibit other HTN-BRD4 from formation of new ternary complex. Kinetic of OGTAC-4 induced O-GlcNAcylation also correspond with this interpretation (FIG. 4D): 640 nM of OGTAC-4 induced O-GlcNAcylation within 2 h and the induction was increasing overtime, peaking at our longest time point, 24 h. This suggested the catalytic characteristic of OGTAC-4 enables it being used at low concentration for longer time.

### LC-MS/MS Identify O-GlcNAcylation Sites of BRD4 Induced by OGTACs

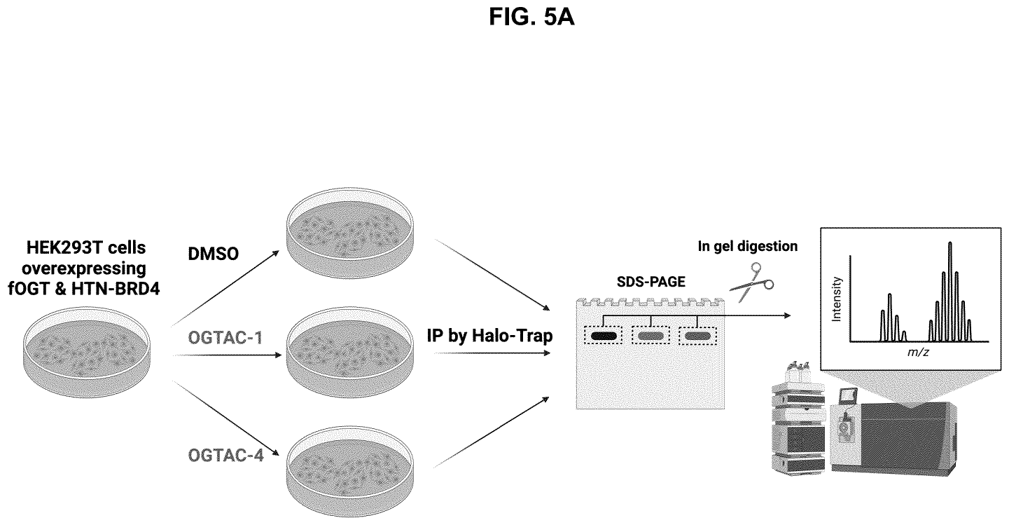

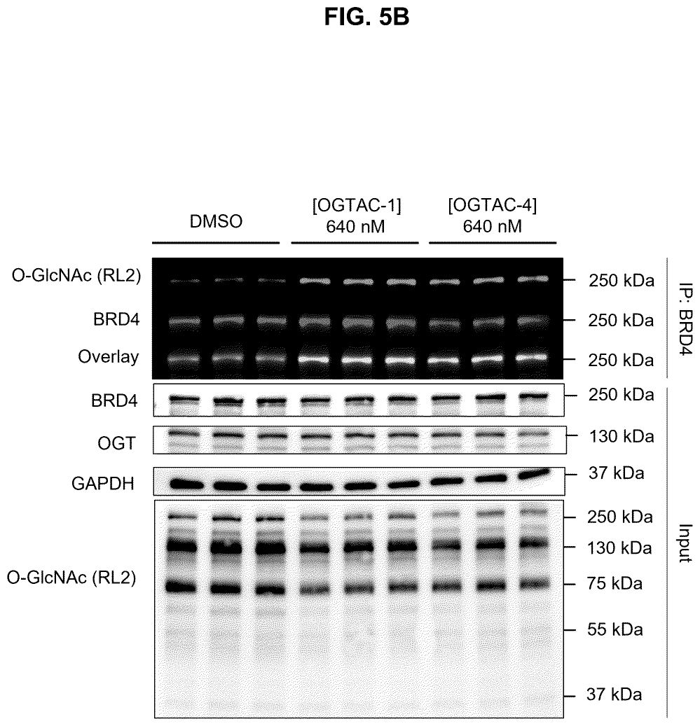

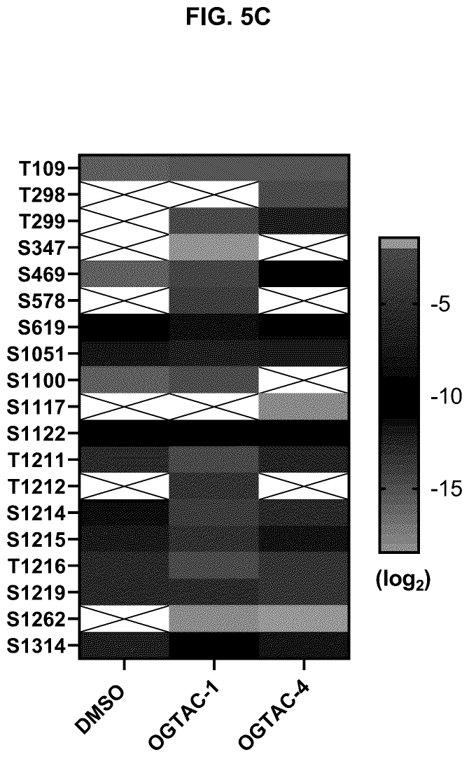

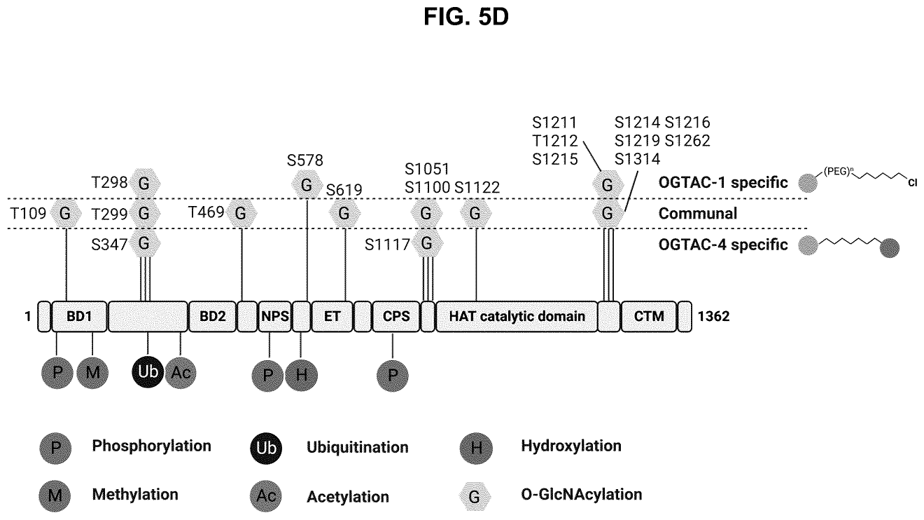

With two effective OGTACs for HTN-BRD4 in hand, we mapped the glycosites they induced and investigated the site selectivity between them using protein Liquid Chromatography with tandem mass (LC-MS/MS). In these experiments, we transfected HEK293T cells with HTN-BRD4:fOGT=20:1 for 24 h, then treat cells with 1) DMSO, 2) 640 nM of OGTAC-1 or 3) 640 nM OGTAC-4 for 4 h. HTN-BRD4 proteins were enriched by HaloTrap agarose from cell lysates and submitted to SDS-PAGE. Target bands at HTN-BRD4 molecular weight level were cut out and in gel digested by later O-GlcNAc sites identification by protein LC-MS/MS (FIG. 5A). In parallel with MS detection, we also used immunoblotting to verify the induction of BRD4 O-GlcNAcylation and their specificity (FIG. 5B).

Enhancing Utility of OGTAC Strategy by a Truncated fOGT Construct

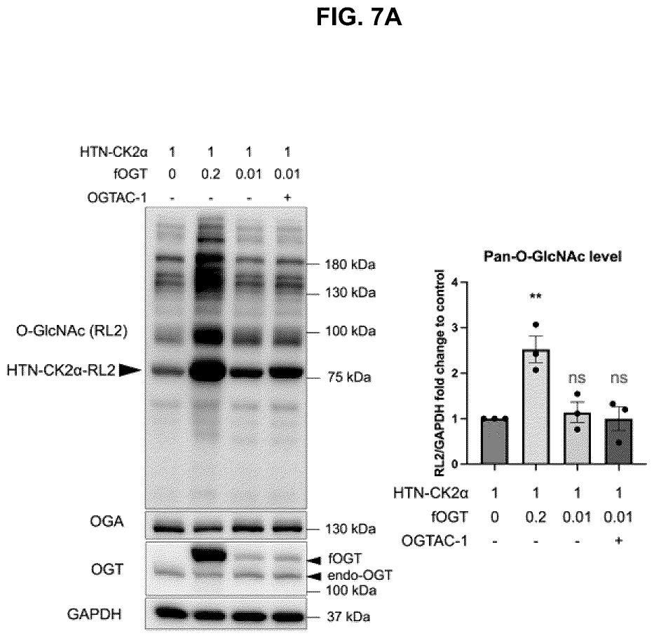

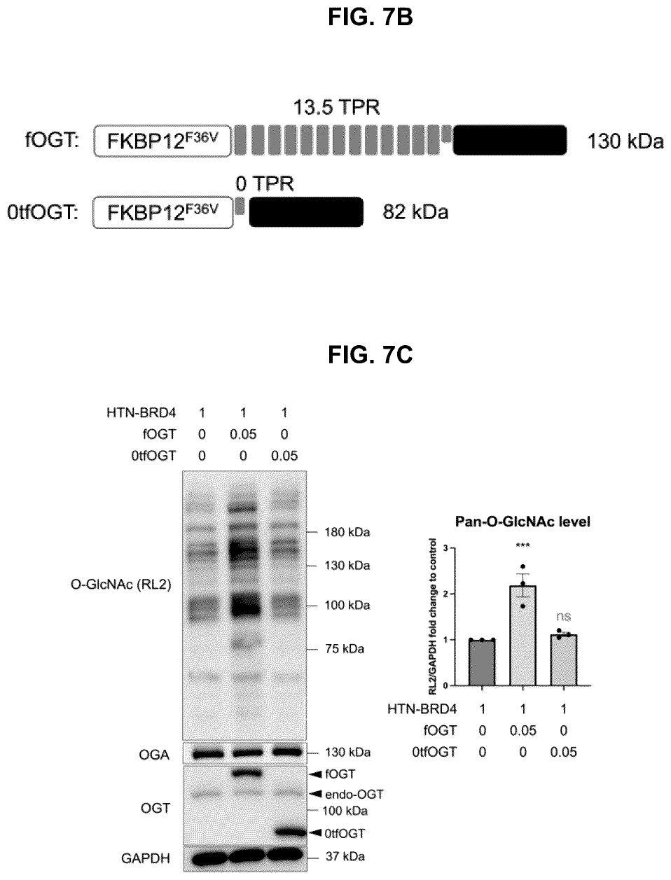

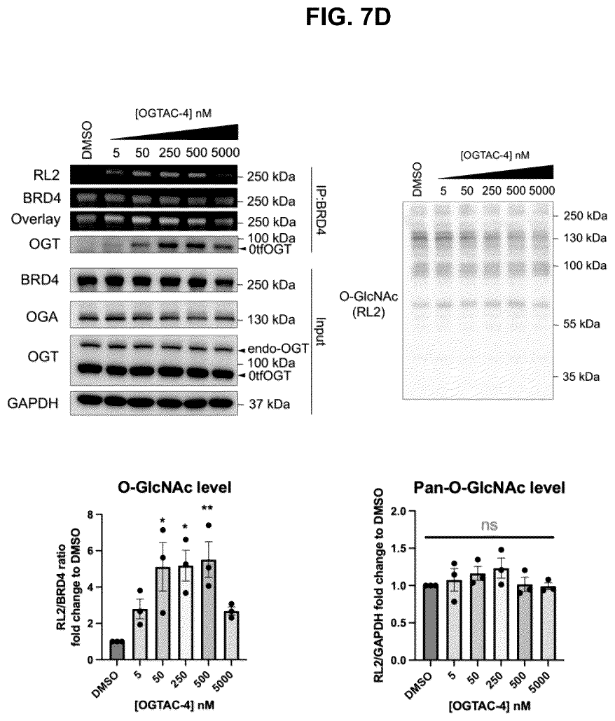

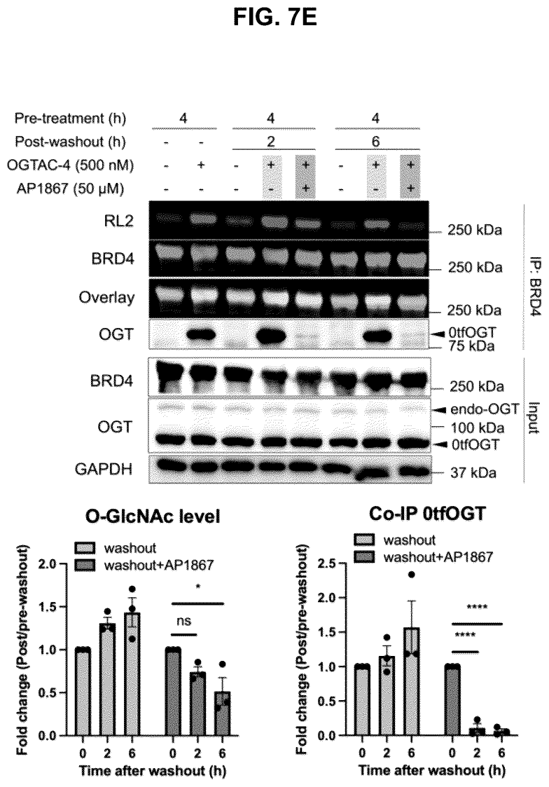

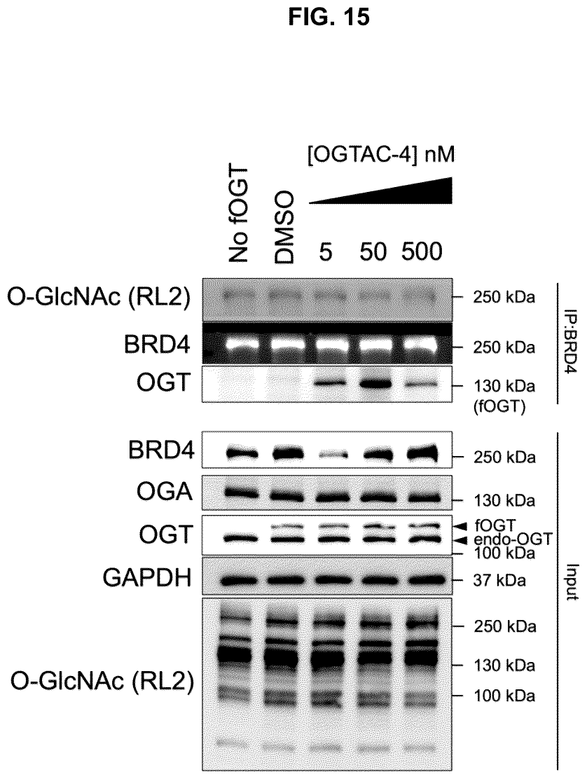

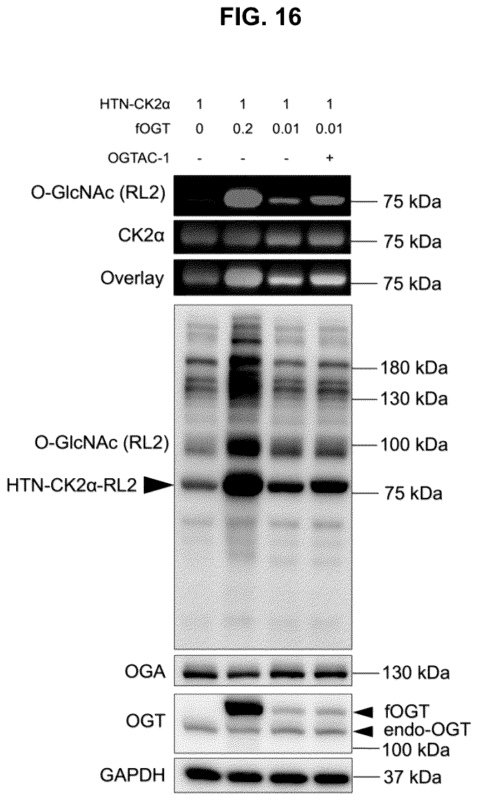

After illustrating the potency and generality of the OGTAC strategy, we then assessed its specificity towards the global O-GlcNAc proteome. We quantified the total pan-O-GlcNAc level (except for the specific band for target POI) using immunoblotting. For well-validated OGT substrates such as CK2α relatively low fOGT protein amount is sufficient to facilitate the inducing effect of our OGTAC-1. The pan-O-GlcNAc level did not significantly change under this condition (FIG. 7A and FIG. 16); however, for BRD4, which is a controversial OGT substrate. HTN-BRD4:fOGT=1:0.01 condition is not sufficient for OGTAC to exert inducing effects even though ternary complex was formed (FIG. 15). We hypothesize that an optimal threshold of OGT protein levels is required for efficient O-GlcNAc transfer to BRD4. To address the trade-off between potency and specificity on BRD4, we developed a truncated fOGT system. The tetratricopeptide repeat (TPR) domain of fOGT is responsible for substrate recognition; truncation of TPR domain can reduce its activity towards protein substrates. We therefore constructed and compared several truncated fOGT (tfOGT) plasmids.

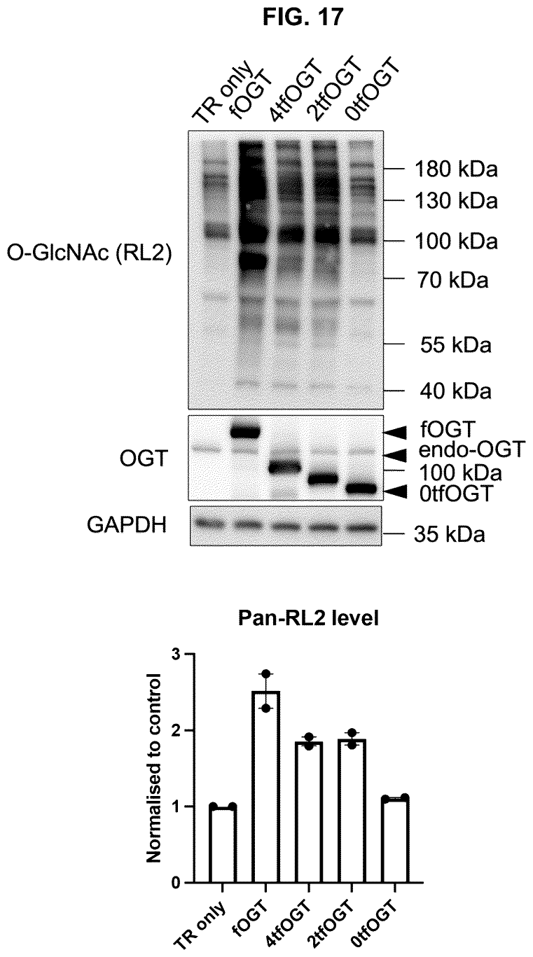

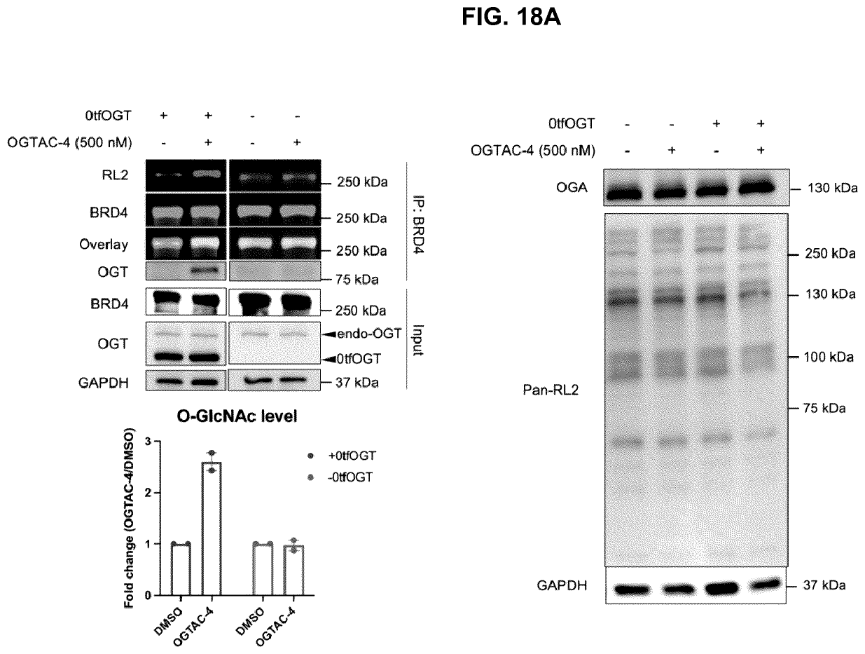

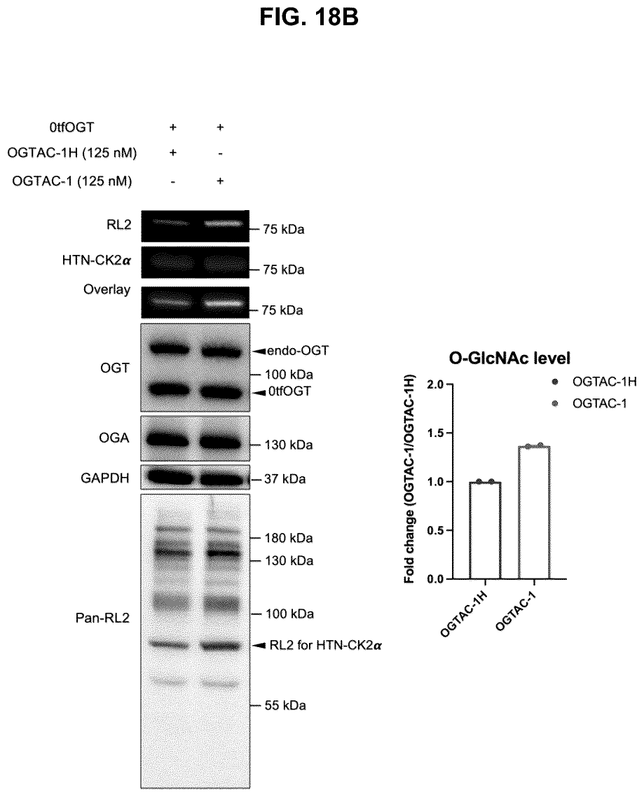

Among them, 0tfOGT showed minimum global O-GlcNAc level elevation even at high expression level (FIG. 7C and FIG. 17). We next applied OGTAC-4 to the 0tfOGT:HTN-BRD4 system, resulting in an 5-fold increase in O-GlcNAc level on BRD4. This result confirms that OGTAC-4 effectively leverages the activity of truncated fOGT to induce BRD4 O-GlcNAcylation without disruption of global O-GlcNAc level (FIG. 7D). Futhermore, we also verified that the inducing effect of OGTAC-4 is dependent on 0tfOGT (FIGS. 18A and 18B).

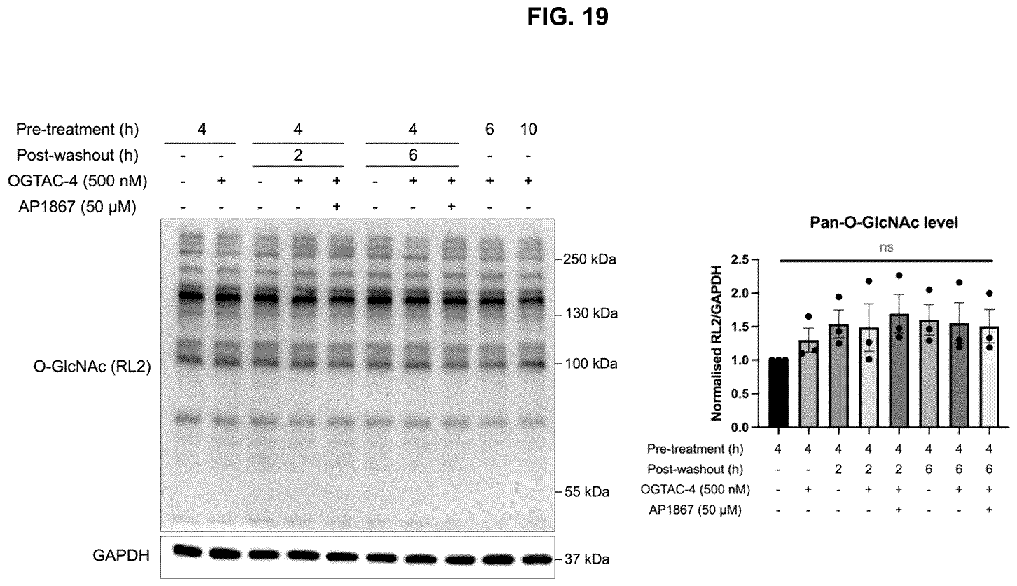

To assess the potential application of our OGTAC strategy in downstream biological studies, we investigated the reversibility of OGTACs. In a washout study using OGTAC-4 as an example, O-GlcNAc induction and ternary complex formation persisted post-washout, attributable to the high affinity of the JQ1 and AP1867 moieties for BRD4 and 0tfOGT, respectively. However, when AP1867 was introduced, a marked decrease in ternary complex formation occurred within 2 hours, indicating that AP1867 can effectively disrupt the tight complex between 0tfOGT and BRD4 (FIG. 7E). Additionally. the O-GlcNAc level of BRD4 returned to baseline after 6 hours of AP1867 treatment, confirming the reversibility of the induction effect (FIG. 7E). Notably, the global O-GlcNAc levels remained unchanged during the reversibility study (FIG. 19)

We envision that this tfOGT system would be a versatile alternative method for inducing and investigating the O-GlcNAcylation of neo-substrates of OGT with high specificity, potency, and reversible control.

### Discussion

Here, we developed the first small molecule-based technology, OGTACs for protein- and site-specific induction of O-GlcNAcylation in live cells. In this technology, OGTAC-1 represents the OGTACs that covalently link and induce O-GlcNAcylation of Halotag-fused POIs by recruiting fOGT; while OGTAC-4 demonstrates BRD4 specific O-GlcNAcylation by recruiting fOGT with sub-stoichiometric catalytic activity. Both OGTAC-1 and OGTAC-4 are submicromolar efficacy O-GlcNAcylation inducers which reveal the characteristics of bifunctional molecules, including promotion of ternary complex and hook effect at excessive stoichiometry against POIs. Furthermore, the length of linker is critical for the potency of OGTACs, and this potency is not directly linked to the formation of ternary complex, which was considered as a golden role for chemical induced proximity.

Current detection methods for protein O-GlcNAcylation are still insensitive and laborious. IP using POI antibody followed by immunoblot using RL2/CTD110.6 antibody is most widely acknowledged. Considering the low throughput of this IP-WB method, we firstly developed two cell based binding assays to validate the OGTACs target engagement and narrow down the effective treatment concentration and time. The pulse-chase assay was developed to verify the OGTACs, which are heterobifunctional molecules that contain haloalkane, can still covalently linked to Halotag-fused protein target in cells. We noted that linker can significantly affect this binding kinetics; longer PEG linker reduced the reaction rate. CETSA demonstrated the direct but not non-covalent binding of OGTACs to fOGT.

Comparing the results from target engagement assays with the assessment of O-GlcNAcylation induction effects, we obtained a deeper understanding of the characteristics of small molecule-induced O-GlcNAcylation. OGTAC-1 prevails in OGTAC-1,2,3 in both binding assays as well as O-GlcNAcylation inducing effects. This indicates that a higher binding affinity to both POI and fOGT might enhance occurrence of the O-GlcNAc transfer reaction. What's more, we noticed that although OGTAC-2 and 3 promoted stable ternary complex, they still showed poor O-GlcNAcylation inducing effects. This result suggested that similar as PROTAC, the linker in OGTACs can significantly affect their potency, probably due to the different conformation they would lead to. The longer length of linker in the OGTACs enable them to recruit individual targets with the respective binder more freely, but also resulting in farther distance between the active site of fOGT and halo-POIs. This phenomenon may be more obvious on the following two scenarios: 1) the bifunctional molecule binding with high affinity. In OGTACs we developed here, the binding motif for fOGT. AP1867 K_{d}=1.8 nM to FKBP12^{F36V}; the other side is covalent linkage to Halotag-POI, rendering an even stronger binder. These hyper-potent binders in OGTACs induced the proximity between fOGT and Halotag-POI and lock their relative position. The distance between them highly depended on the linker length of OGTACs. Longer distance between two proteins caused by longer linker could be hamper the O-GlcNAc transfer from fOGT active site to Halo-POIs. 2) the two proteins being induced together are not natural interactive partners. Although OGT O-GlcNAcylates thousands of cellular proteins, BRD4 has not been reported as a direct substrate of it. This indicates that OGTACs may only force two proteins in proximal localisation but not stimulate any interaction. OGTAC-1,2,3 irreversibly modified Halotag on BRD4, tagging the HTN-BRD4 protein with AP1867 which recruits fOGT; This binary interaction is only based on AP1867-FKBP12^{F36V} binding without any cooperative effects between OGT and BRD4 protein-protein interaction. Comparatively, OGTAC-4 binds to BD1 and BD2 domain of BRD4, which could lead to different ternary complex conformation. The non-covalent binding mode on both sides may also enable the BRD4 and TOGT interacting more freely. Furthermore, considering AP1867 is a low nanomolar binder FKBP12^{F36V} (K_{d}=1.8 nM)²⁷, JQ1 motif (K_{d}=49.0 nM for BD1, 90.1 nM for BD2)²⁸ is more likely to dissociate and allow neo BRD4 proteins to dock in and initiate a new O-GlcNAc transfer process. As a result, OGTAC-4 could act as catalyst, therefore a low concentration was sufficient for achieving similar effects with OGTAC-1 and prolonged treatment is favoured to further enhance the potency.

### Materials and Methods

### Construct of HTN-BRD4

SEQ ID NO:16. HaloTag fragment is in bold. The 1051-1224 fragment (where most O-GlcNAc modification occurs) is underlined.

MAEIGTGFPFDPHYVEVLGERMHYVDVGPRDGTPVLFLHGNPTSSYVWRN
IIPHVAPTHRCIAPDLIGMGKSDKPDLGYFFDDHVRFMDAFIEALGLEEV
VLVIHDWGSALGFHWAKRNPERVKGIAFMEFIRPIPTWDEWPEFARETFQ
AFRTTDVGRKLIIDQNVFIEGTLPMGVVRPLTEVEMDHYREPFLNPVDRE
PLWRFPNELPIAGEPANIVALVEEYMDWLHQSPVPKLLFWGTPGVLIPPA
EAARLAKSLPNCKAVDIGPGLNLLQEDNPDLIGSEIARWLSTLEISGEPT
TEDLYFQSDNAIASEFMSAESGPGTRLRNLPVMGDGLETSQMSTTQAQAQ
PQPANAASTNPPPPETSNPNKPKRQTNQLQYLLRVVLKTLWKHQFAWPFQ
QPVDAVKLNLPDYYKIIKTPMDMGTIKKRLENNYYWNAQECIQDFNTMFT
NCYTYNKPGDDIVLMAEALEKLFLQKINELPTEETEIMIVQAKGRGRGRK
ETGTAKPGVSTVPNTTQASTPPQTQTPQPNPPPVQATPHPFPAVTPDLIV
QTPVMTVVPPQPLQTPPPVPPQPQPPPAPAPQPVQSHPPIIAATPQPVKT
KKGVKRKADTTTPTTIDPIHEPPSLPPEPKTTKLGQRRESSRPVKPPKKD
VPDSQQHPAPEKSSKVSEQLKCCSGILKEMFAKKHAAYAWPFYKPVDVEA
LGLHDYCDIIKHPMDMSTIKSKLEAREYRDAQEFGADVRLMFSNCYKYNP
PDHEVVAMARKLQDVFEMRFAKMPDEPEEPVVAVSSPAVPPPTKVVAPPS
SSDSSSDSSSDSDSSTDDSEEERAQRLAELQEQLKAVHEQLAALSQPQQN
KPKKKEKDKKEKKKEKHKRKEEVEENKKSKAKEPPPKKTKKNNSSNSNVS
KKEPAPMKSKPPPTYESEEEDKCKPMSYEEKRQLSLDINKLPGEKLGRVV
HIIQSREPSLKNSNPDEIEIDFETLKPSTLRELERYVTSCLRKKRKPQAE
KVDVIAGSSKMKGFSSSESESSSESSSSDSEDSETEMAPKSKKKGHPGRE
QKKHHHHHHQQMQQAPAPVPQQPPPPPQQPPPPPPPQQQQQPPPPPPPPS
MPQQAAPAMKSSPPPFIATQVPVLEPQLPGSVFDPIGHFTQPILHLPQPE
LPPHLPQPPEHSTPPHLNQHAVVSPPALHNALPQQPSRPSNRAAALPPKP
ARPPAVSPALTQTPLLPQPPMAQPPQVLLEDEEPPAPPLTSMQMQLYLQQ
LQKVQPPTPLLPSVKVQSQPPPPLPPPPHPSVQQQLQQQPPPPPPPQPQP
PPQQQHQPPPRPVHLQPMQFSTHIQQPPPPQGQQPPHPPGQQPPPPQPAK
PQQVIQHHHSPRHHKSDPYSTGHLREAPSPLMIHSPQMSQFQSLTHQSPP
QQNVQPKKQELRAASVVQPQPLVVVKEEKIHSPIRSEPFSPSLRPEPPKH
PESIKAPVHLPQRPEMKPVDVGRPVIRPPEQNAPPPGAPDKDKQKQEPKT
PVAPKKDLKIKNMGSWASLVQKHPTTPSSTAKSSSDSFEQFRRAAREKEE
REKALKAQAEHAEKEKERLRQERMRSREDEDALEQARRAHEEARRRQEQQ
QQQRQEQQQQQQQQAAAVAAAATPQAQSSQPQSMLDQQRELARKREQERR
RREAMAATIDMNFQSDLLSIFEENLF

### Cell Cultures, DNA Constructs, and Reagents

HEK293T cells and HeLa cells (kind gift from Prof. Sze Lok Cheng, CUHK) were maintained and cultured at 37° C. with 5% CO₂ in Dulbecco's modified Eagle's medium (DMEM) supplemented with 10% fetal bovine serum and 1% penicillin and streptomycin.

FKBP12^{F36V}-OGT vector was a gift from Walker's group (Harvard Medical School).

HTN-BRD4 plasmids were constructed by subclone from pcDNA4-TO-HA-Brd4FL (addgene: #31351), then homologous recombination (Vazyme C115) into pHTN HaloTag® CMV-neo Vector (Promega G7711).

HTN-CK2α plasmids were constructed by subclone from pDB1 (CK2αlpha) (addgene: #27083), then homologous recombination into pHTN HaloTag® CMV-neo Vector.

HTN-EZH2 plasmids were constructed by subclone from 3XMyc-His₆-EZH2 plasmid from Prof. YangChao Chen (CUHK), then homologous recombination into pHTN HaloTag® CMV-neo Vector.

Transient transfection was conducted according to the manufacture's protocol using Lip2000 TR (AboRo, RL0401).

Plasmids site-directed mutagenesis was conducted according to the manufacture's protocol using Mut Express II Fast Mutagenesis Kit V2 (Vazyme. (214)

### Primers for Mutagenesis

| No | Primer name | Sequence (5′ to 3′) |
|---|---|---|
| 1 | 4TPR(327-1046)- | CACAGCAGACTCTCTGAATAACCTAGCCAATAT (SEQ |
| | fOGT fwd | ID NO: 17) |
| 2 | 4TPR(327-1046)- | TTCAGAGAGTCTGCTGTGCTGTCGGCCACGTTG |
| | fOGT rev | (SEQ ID NO: 18) |
| 3 | 2TPR(395-1046)- | ACAGCTGATGCCTACTCTAATATGGGAAACACT (SEQ |
| | fOGT fwd | ID NO: 19) |
| 4 | 2TPR(395-1046)- | AGAGTAGGCATCAGCTGTGCTGTCGGCCACGTT |
| | fOGT rev | (SEQ ID NO: 20) |
| 5 | 0TPR(463-1046)- | ACAGCACACCTGATGCTTATTGTAACTTGGCTC (SEQ |
| | fOGT fwd | ID NO: 21) |
| 6 | 0TPR(463-1046)- | AAGCATCAGGTGTGCTGTCGGCCACGTTGCCCA |
| | fOGT rev | (SEQ ID NO: 22) |
| 7 | HTN-BRD4 | CACCACAAGGAGCAGTTCCGCCGCGCCGCTCGG |
| | Δ1051-1224 rev | (SEQ ID NO: 23) |
| | fwd | |
| 8 | HTN-BRD4 | GAACTGCTCCTTGTGGTGCCGGGGTGAATGGTG |
| | Δ1051-1224 rev | (SEQ ID NO: 24) |
| 9 | HTN-BRD4 | GgcggacccctacgcaACCGGTCACCTCCGCGAA (SEQ ID |
| | S1051A/ S1055A | NO: 25) |
| | fwd | |
| 10 | HTN-BRD4 | TtgcgtaggggtccgcCTTGTGGTGCCGGGGTGA (SEQ ID |
| | S1051A/ S1055A | NO: 26) |
| | rev | |
| 11 | HTN-BRD4 | ccctagtgcagaagcatccggccACCCCCTCCTCCACAGCC |
| | 1201A/1204A/121 | (SEQ ID NO: 27) |
| | 1A fwd | |
| 12 | HTN-BRD4 | atgcttctgcactagggcggcccaggcGCCCATGTTCTTGATTTT |
| | 1201A/1204A/121 | CAGG (SEQ ID NO: 28) |
| | 1A rev | |
| 13 | HTN-CK2α S347A | TCgccagcGCCAATATGATGTCAGGGATTTCTT (SEQ |
| | fwd | ID NO: 29) |
| 14 | HTN-CK2α S347A | TCATATTGGCgctggcGACGGGCGTACTGCCCCC (SEQ |
| | rev | ID NO: 30) |
| 15 | HTN-CK2α | TCgccgccGCCAATATGATGTCAGGGATTTCTT (SEQ ID |
| | S347A/S348A | NO: 31) |
| | fwd | |
| 16 | HTN-CK2α | TCATATTGGCggcggcGACGGGCGTACTGCCCCC |
| | S347A/S348A rev | (SEQ ID NO: 32) |

All primers were synthesized by TechDragon Limited. The open reading frame of mutant plasmids were sequenced by BGI TECH SOLUTIONS (BEIJING LIUHE) CO., LIMITED. before conducting experiments.

### Pulse-Chase Assay

1×10⁵ HEK293T cells were seeded in each well of 12 well plate. After cells attached, transfection reagents and plasmids were prepared and added in DMEM. After 24 h transfection, media were replaced by fresh warm complete media with DMSO or compounds. After certain time of OGTACs treatment, media in wells were replaced by fresh warm media with 5 μM rhodamine ligand. The cells were incubated in 37° C.′ for 15 mins. Then, media were aspirated and 100 μL IP lysis buffer (Thermo Scientific 87788) supplemented with 20 μM OGA inhibitor (Thiamet G, Bidepharm BD571819) and 100X protease inhibitor (MedChemExpress. HY-K0010) (referred as complete IP lysis buffer in the later paragraph) was added to each well. The lysate was incubated on ice for 20 min, spun down at 14,000 RPM at 4° C. for 15 mins, and the supernatant were collected for bicinchoninic acid (BCA) assay and normalized to a final 2 mg/mL. The lysates were denatured by SDS-loading buffer (Biorad #1610747) and 10 μL of each sample was loaded in SDS-PAGE. After running, in gel fluorescence at rhodamine channel (545/575 nm) was analysed by Bio-Rad ChemiDoc MP Imaging System. After image, the gel was transferred to PVDF membrane to check equal loading using anti-GAPDH antibody.

### CETSA

HEK293T cells (2.5×10⁶ cells) were seeded in 10 cm culture dish overnight for attachment. On the next day, cells were transfected with HTN-BRD4:fOGT=1:0.05 plasmid for 24 hours until ˜90% confluence. After treatment with 5 μM compounds or DMSO, cells were collected and washed with PBS. Subsequently, cells were re-suspended in 500 μL PBS and equally divided into 50 μL aliquots. The tubes were subjected to heat challenge at 37-53° C. for 5 mins, followed by cooling on ice. Cells were lysed by three repeated freeze-thaw cycles with liquid nitrogen and water bath. Lastly, cells were centrifuged at 14000 RPM for 15 mins and supernatants were collected for Western Blot analysis.

### Immunoprecipitation

For each IP reaction, 2.2×10⁶ HEK293T cells were seed in 10 cm dish. After cells attached, transfection reagents and plasmids were prepared and added in DMEM. After 24 h transfection, media were replaced by fresh warm complete media with DMSO or probes. For washout study, after 4 h treatment, media with DMSO or OGTAC-4 (500 nM) were replaced by fresh media with DMSO or AP1867 (50 μM) for further incubation. After certain time of OGTACs treatment, cells were collected by cold PBS and lysed by 200 μL complete IP lysis buffer. The lysate was incubated on ice for 20 min, spun down at 14,000 RPM at 4° C. for 15 mins, and the supernatant were collected for bicinchoninic acid (BCA) assay and normalized to a final 2 mg/mL. For input, 20 μL of diluted sample was reacted with equal volume of 10 μM TMR ligand in IP lysis buffer and rotate gently at RT for 15 mins. The remaining samples were subjected to protein A/G magnetic beads (MedChemExpress, HY-K0202), which pre-binding with protein target protein antibody. For samples for LC-MS/MS or HTN-BRD4 mutant constructs. Halo-Trap Magnetic Agarose (Proteintech, otma) was used to incubate with protein lysates. After gentle rotation at 4° C. for at least 16 h, the beads were washed with IP lysis buffer and boiled in 40 μL 2×SDS-loading buffer (Biorad #1610747) for 5 mins to elute proteins from beads. Eluted samples were directly subjected into WB analysis. To get stronger signal of RL2, we used anti-mouse-HRP (Cell signaling, #7076) as secondary antibody for RL2 and anti-rabbit-DyLight 488 (Invitrogen, #35552) as secondary antibody for total target proteins.

### Chemoenzymatic Labelling and Mass Shift Assay

For each chemoenzymatic labelling reaction. 2×10⁵ HEK293T cells were seed to each well of 6 well plate. After cells attached, transfection reagents and plasmids were prepared and added in DMEM. After 24 h transfection, media were replaced by fresh warm complete media with DMSO or probes. After certain time of OGTACs treatment, cells were collected by cold PBS and lysed by 200 μL RIPA lysis buffer (Thermo Scientic 89901) supplemented with 20 μM OGA inhibitor (Thaimet G, Bidepharm BD571819), 100X protease inhibitor (MedChemExpress, HY-K0010) and 1 μL/mL BevoZonase (Beyotime, D7121). The supernatant of lysate was then quantified by bicinchoninic acid (BCA) assay and normalized to 2 mg/mL with 1% SDS in buffer. Then. lysate was reduced by DTT (20 mM) at 58° C. for 1 h and alkylated by IAA (90 mM) for 40 mins in dark. For 100 μL lysate, added in sequence with vortex: 400 μL MeOH, 100 μL CHCl₃, 300 μL H₂O. The protein pellet was then washed with MeOH 500 μL and redissolved in 1% SDS GalT buffer. The following reagents were add in sequence: Protein lysate: 40 μL; H₂O: 49 μL; Label buffer: 80 μL; MnCl₂: 11 μL; UDP; GalNAz: 10 μL; Protease inhibitor: 2 μL; Gal-T Y289L: 5 μL; Final volume: 200 μL. The reaction was incubated at 4° C. for 20 h with gentle rotation. The proteins were then precipitated, washed by MeOH and redissolved in 1% SDS GalT buffer, and reacted with DBCO-PEG5k (final concentration 1 mM) for 5 mins at 95° C. The protein were then precipitated, washed by MeOH and redissolved in 1 ×SDS-loading buffer for western blot analysis.

### Western Blotting and Antibody

In general, cells (from 12 well plate) were lysed by 80 μl RIPA lysis buffer (Thermo Scientic 89901) supplemented with 20 μM OGA inhibitor (Thaimet G, Bidepharm BD571819) and 100X protease inhibitor (MedChemExpress, HY-K0010), incubated on ice for 20 min, spun down at 14,000 RPM at 4° C. for 15 mins, and the supernatant were collected for bicinchoninic acid (BCA) assay and normalizedeto a final 2 mg/mL concentration. About 30 μg of protein samples were loaded for sodium dodecyl sulfatepolyacrylamide gel electrophoresis (SDS-PADE) and blotted with indicated antibodies. Antibodies used in this study are as follow:

| | | Dilution | |
|---|---|---|---|
| Antibody | Brand | (application) | |
| RL2 | Abcam (ab2739) | 1:1000 | (WB) |
| Anti-BRD4 | Cell Signalling | 1:2000 | (WB) |
| | Technology (CST) | 1:100 | (IP) |
| | (13440S) | | |
| Anti-BRD4 (for mutant) | Abclonal (A12677) | 1:1000 | (WB) |
| Anti-OGT | CST (D1D8Q) | 1:1000 | (WB) |
| Anti-CK2α | CST (2656) | 1:1000 | (WB) |
| Anti-EZH2 | CST (5246) | 1:2000 | (WB) |
| | | 1:100 | (IP) |
| Anti-CK2α | Proteintech (10992-1-AP) | 1:50 | (IP) |
| Anti-GAPDH | Santa Cruz (sc-47724) | 1:3000 | (WB) |
| Anti-HaloTag | Promega (G9211) | 1:1000 | (WB) |
| Goat anti-Mouse-HRP | CST (7076) | 1:6000 | (WB) |
| Goat anti-rabbit-HRP | CST (7074) | 1:6000 | (WB) |
| Goat anti-rabbit-DyLight | Invitrogen (35552) | 1:6000 | (WB) |
| 488 | | | |
| IRDye ® 680RD Goat | LI-COR (926-68071) | 1:20000 | (WB) |
| anti-Rabbit IgG (H + L) | | | |
| Normal rabbit IgG | CST (2729) | 1:100 | (IP) |

### Mass Spectrometry

For MS sample preparation, cell pellets were treated and collected same as the method in immunoprecipitation. HaloTrap Magnetic Agarose (proteintech, otma) was separated as 25μL aliquot for each reaction. The beads were washed three times with 500 μL IP lysis buffer, followed by complete removal of supernatant. Then, cell lysates were added to each tubes and incubated at 4° C. for 4 h with gentle rotation. After incubation, beads were intensively washed with IP lysis buffer and eluted in 2 ×SDS-loading buffer heated at 95° C. for 5 mins. The samples were submitted to SDS-PAGE and stained with Coomassie brilliant blue solution, and the bands around 250 kDa were cut out, washed three times by ddH₂O. destained in 100 μL destaining buffer for 20 mins at 25° C. and this process was repeated once. The gel was then washed by 100% ACN for 15 mins and dried. Then tris(2-carboxyethyl) phosphine (TCEP) was added to reduce protein for 30 mins at 25° C., followed by addition of iodoacetamide (IAA) solution for 30 mins reaction at 25° C. in dark. The supernatant was discarded and wash by 100% ACN, freeze dried. The sample was then digested by Trypsin solution for 20 h at 37° C. After centrifugation, supernatant was collected and digested peptides were extracted by the following steps. Extraction solution was added and incubated for 20 mins at 25° C.; this step was repeated once and 100% ACN was added to extracted again. All extracted fractions were combined and freeze dried and desalt by C18 columns. The elutes from columns were freeze dried and injected to MS.

### Mass Spectrometry Acquisition Procedures

LC-MS/MS data was collected by timsTOF Pro2 mass spectrometer coupled with a nanoElute UPLC system (Bruker Daltonics, Bremen, Germany). The peptides were dissolved in phase A (0.1% formic acid in water). 100 ng of peptides were analysed by C18 column (Aurora Series. 75 μm×25 cm, Ionopticks, Victoria, Australia) with the gradient set as: 0-48 min, 4-18% solvent B (0.1% formic acid in ACN); 48-55 min, 18-35% B; 55-57 min, 35-95% B; 57-60 min, 95% B with flow rate at 300 nL/min. Peptides were analysed using DDA mode by LC-MS/MS. The parameters set as follows: scan range (m/z)=300-1500; tims scan range or 1/K0 range (V·s/cm2)=0.75-1.35; MSI resolution=60,000; Target Intensity=100000; Intensity Threshold=2500; number of PASEF MS/MS scans=6; Total cycle time=1.16s; charge range=2-5; Isolation Width: 2 m/z (when <800m/z), 3 m/z (when >800 m/z);

### Mass Spectrometry Data Analysis

The raw data were processed using PEAKS Studio (version 11, Bioinformatics Solutions Inc., Waterloo, Canada) against Homo sapiens proteome in UniProt/SwissProt human (Homo sapiens) protein database plus our construct (HTN-BRD4). The parameters set as follows: Precursor Mass Error Tolerance: 20.00 ppm, Fragment Mass Error Tolerance: 0.05 Da, Enzyme: Trypsin, Max Missed Cleavage: 2, Digest Mode: Specific, Peptide Length Range: 5-45, Max Variable PTM per Peptide: 3, Fixed Modifications: Carbamidomethylation (+57.02), Variable Modifications: HexNAcylation (ST) (+203.08), Oxidation (M) (+15.99).Database: Homo sapiens proteome (20387 proteins) plus our construct (HTN-BRD4), Taxonomy: all species, Searched Entries: 1, Deep Learning Boost: Yes, Report Filter: higher than 1% false discovery rate (FDR) <1% and protein unique peptides >=2; the O-GlcNAc modification specific-peptide-to-spectrum match (PSM) using characteristic peak with >1% ion intensity. The O-GlcNAcylation modification occupancy was calculated by detected modified peptides intensity divided by detected total peptide intensity for each sample.

### Chemical Synthesis

NMR spectra were acquired on Bruker 400 & 500 NMR spectrometer, running at 400 MHz for ¹H and Bruker 500 NMR spectrometer at 126 MHz for ¹³C respectively. ¹H NMR spectra were recorded at 400 MHz in CDCl₃, using residual CHCl₃ as the internal standard. ¹³C NMR spectra were recorded at 126 MHz in CDCl₃ using residual CHCl₃ as the internal standard. Reactions were monitored by thin layer chromatography and the products were purified using preparative thin layer flash chromatography (ALUGRAM Xtra, 818333). Mass spectrometry was performed on Agilent LC-MS/MS system consisted of two Agilent 1290 series pumps and auto-sampler, coupled with 6430 triple quadrupole mass spectrometer equipped with and ESI source (Agilent Technologies, Inc., Santa Clara, CA, USA). Unless otherwise noted, analytical grade solvents and commercially available reagents were used without further purification. Unless otherwise noted, chemical starting materials are purchased from Bide pharm without further purification.

### (R)-1-(3-(2-((2-(2-((6-chlorohexyl)oxy)ethoxy)ethyl)amino)-2-oxoethoxy)phenyl)-3-(3,4-dimethoxyphenyl)propyl (S)-1-((S)-2-(3,4,5-trimethoxyphenyl)butanoyl) piperidine-2-carboxylate (OGTAC-1) and analogues

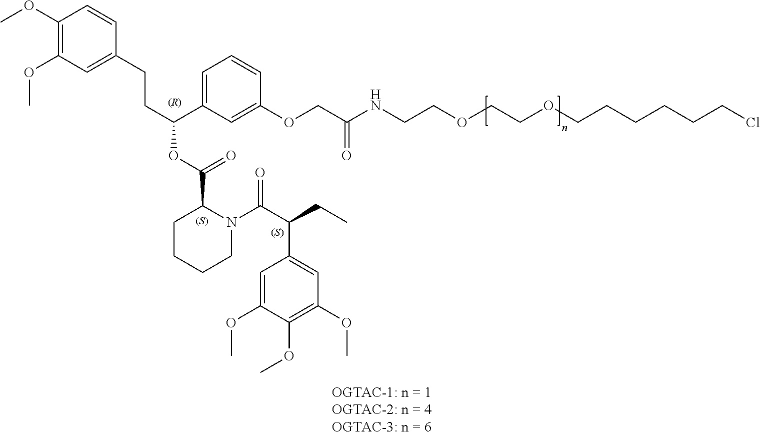

General method: Synthesis of OGTAC-1/2/3 were adapted from literature.² Take OGTAC-1 as an example, the 2-[3-[(1R)-3-(3,4-dimethoxyphenyl)-1-[(2S)-1-[(2S)-2-(3,4,5-trimethoxyphenyl)butanoyl]piperidine-2-carbonyl]oxy-propyl]phenoxy]acetic acid (55.2 mg, 0.079 mmol) (AP1867, synthesized according to literature^{3,4}) was dissolved in DMF (1.5 mL), 1-[Bis(dimethylamino)methylene]-1H-1,2,3-triazolo[4,5-b]pyridinium 3-oxide hexafluorophosphate (HATU) (35 mg, 0.1 mmol), and DIPEA (50 μL, 0.35 mmol) were added and stirred for 30 minutes. NH₂-PEG₂-C6-Cl (20 mg. 0.1 mmol) was added. The reaction mixture was stirred overnight. The reaction mixture was extracted with ethyl acetate and water, purified on preparative TLC (ALUGRAM Xtra, 818333), and evaporated under vacuum to give product as clear oil (26 mg, 35%). Product confirmed by ESI-MS m/z: 899.6 [M+H]⁺, 921.5 [M+Na]⁺, 937.6 [M+K]⁺,; and ¹H-NMR according to literature.² Other probes are synthesised using same reagents just changing NH₂-PEG₂-C6-Cl to NH₂-PEG₅-C6-Cl, NH₂-PEG₇-C6-Cl, and the products were confirmed by ESI-MS and ¹H-NMR according to literature.².

### (R)-3-(3,4-dimethoxyphenyl)-1-(3-(2-((2-(2-(hexyloxy)ethoxy)ethyl)amino)-2-oxoethoxy)phenyl)propyl(S)-1-((S)-2-(3,4,5- trimethoxyphenyl)butanoyl)piperidine-2-carboxylate

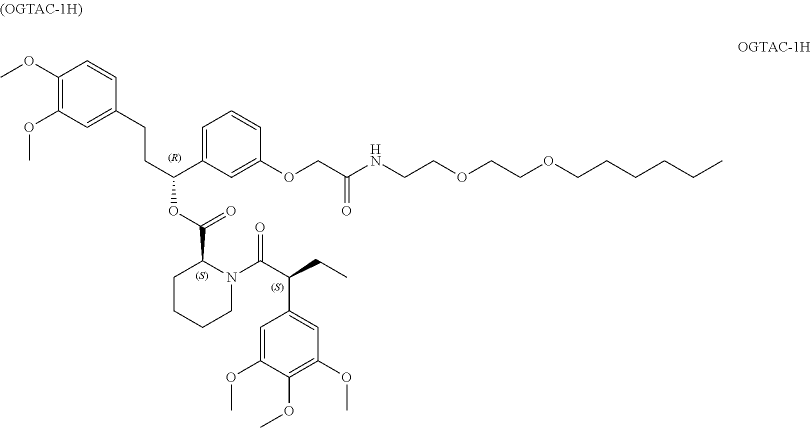

It was synthesized as the general procedures above. AP1867 (56 mg, 0.08 mmol) was dissolved in DMF (2 mL), 1-[Bis(dimethylamino)methylene]-1H-1,2,3-triazolo[4,5-b]pyridinium 3-oxide hexafluorophosphate (HATU) (35 mg. 0.1 mmol), and DIPEA (50 μL, 0.35 mmol) were added and stirred for 30 minutes. NH₂-PEG₂-C6 (34 mg, 0.15 mmol) (BCLP-38, Xi'an Confluore Biological Technology Co., Ltd.) was added. The reaction mixture was stirred overnight. The reaction mixture was extracted with ethyl acetate and water, purified on preparative TLC (ALUGRAM Xtra, 818333), and evaporated under vacuum to give product as clear oil (28 mg, 40%). Product confirmed by ESI-MS m/z: 865.7.6 [M+H]⁺, 887.7 [M+Na]⁺. ¹H-NMR: δ 7.20-7.14 (m, 1H), 6.80-6.74 (m, 3H), 6.69-6.61 (m, 3H), 6.40 (d, J=2.0 Hz, 2H), 5.62 (dd, J=8.2, 5.5 Hz, 1H), 5.47-5.43 (m, 1H), 4.49 (m, 2H), 3.88-3.80 (m, 11H), 3.78 (s, 3H), 3.68 (s, 6H), 3.50-3.62 (m, 12H), 3.43 (td, J=6.7, 1.6 Hz, 2H), 2.84-2.73 (m, 1H), 2.63-2.40 (m, 4H), 2.17-2.01 (m, 3H), 1.76-1.50 (m, 5H), 0.90 (m, 8H); ¹³C NMR: δ 172.77, 170.67, 168.34, 157.39, 153.28, 148.96, 147.44, 142.38, 136.71, 135.40, 133.44, 129.91, 120.27, 119.84, 114.09, 112.85, 111.76, 111.35, 105.05, 104.64, 75.77, 71.67, 70.46, 70.06, 69.80, 67.40, 60.87, 56.38, 56.06, 56.01, 55.93, 52.16, 50.88, 43.58, 38.95, 38.37, 31.77, 31.37, 29.80, 29.67, 28.44, 26.90, 25.85, 25.43, 22.71, 21.03, 14.15, 12.66.

### 4-((2-(2-((6-chlorohexyl)oxy)ethoxy)ethyl)carbamoyl)-2-(6-(dimethylamino)-3-(dimethyliminio)-3H-xanthen-9-yl)benzoate (rhodamine ligand)

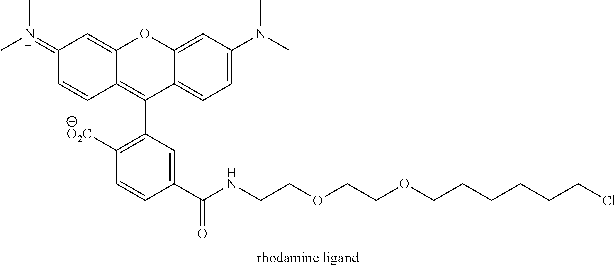

This probe was synthesised following the method described in literature.⁵ 5(6)-TAMRA NHS ester (14 mg. 0.027 mmol) was dissolved in DMF (2 mL) and ten equivalents diisopropyethylamine (DIPEA) (24 μL) was added to the resultant solution. Then NH₂-PEG₂-C6-Cl (10 mg, 0.039 mmol) was added to reaction. The reaction was protected from light and reacted for 8 h. Subsequently, the product was dissolved in water and freeze dried, followed by reconstitution in MeOH. The product was purified by PTLC to get final product as dark red powder (15 mg, 86%). Product confirmed by ESI-MS m/z: 646 [M+H]⁺.

### (R)-1-(3-(2-((6-((tert-butoxycarbonyl)amino)hexyl)amino)-2-oxoethoxy)phenyl)-3-(3,4-dimethoxyphenyl)propyl (S)-1-((S)-2-(3,4,5- trimethoxyphenyl)butanoyl)piperidine-2-carboxylate (AP1867-C6-NHBoc)

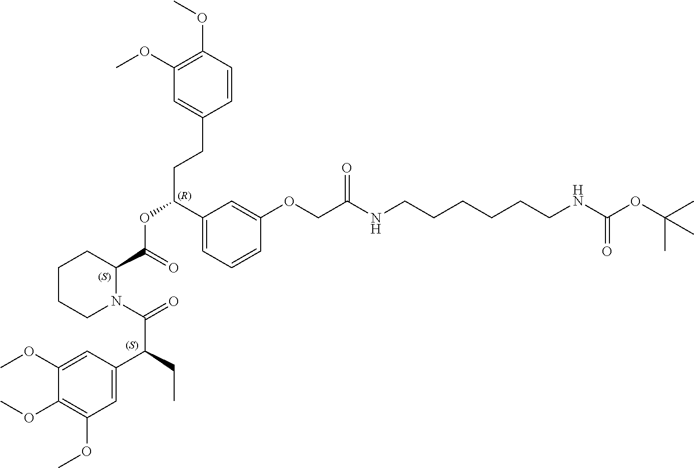

To a round bottom flask (RBF), AP1867 (57.8 mg, 0.08 mmol) was added and dissolved in 2 mL dry DMF. Then DIPEA (70 μL) and HATU (45.6 mg, 0.12 mmol) were added to the solution and stirred for 15 mins. N-Boc-1,6-diaminohexane (25.3 mg, 0.1 mmol) was then added and stirred for another 16 h. The reaction was then partitioned in EtOAc and H₂O, extracted with EtOAc and the dried over Na₂SO₄. The crude product was purified by PTLC (EtOAc: Hexane32 2:1) to have clear oil (33 mg, 51%). ESI-MS m/z: 691 [M+H]⁺(without Boc). ¹H-NMR: δ 7.21-7.14 (m, 1H), 6.83-6.77 (m, 3H), 6.72-6.63 (m, 3H), 6.43 (d, J=2.0 Hz, 2H), 5.67 (dd, J=8.2, 5.5 Hz, 1H), 5.49-5.45 (m, 1H), 4.50 (m, 2H), 3.88-3.81 (m, 10H), 3.78 (s, 3H), 3.68 (s, SH), 3.61 (t, J=6.7 Hz, 1H), 3.40-3.31 (m, 2H), 3.15-3.06 (td, 2H), 2.86-2.76 (t, 1H), 2.65-2.47 (m, 2H), 2.37-2.28 (m, 1H), 2.15-1.98 (m, 4H), 1.79-1.25 (m, 5 H), 1.60 (s, 6H), 1.45 (s, 9H), 0.93 (t, 3H); ¹³C-NMR: δ 172.86, 170.73, 168.32, 157.43, 153.30, 148.99, 147.78, 142.47, 136.73, 135.40, 133.45, 129.95, 120.31, 119.96, 113.65, 113.13, 111.80, 111.38, 105.07, 75.73, 60.90, 56.43, 56.04, 55.97, 52.20, 50.91, 43.60, 40.70, 39.10,38.36, 31.42, 30.06, 29.83, 29.59, 28.45, 26.92, 26.55, 26.42, 25.44, 21.02, 12.86, 12.69.

### (R)-1-(3-(2-((6-(2-((S)-4-(4-chlorophenyl)-2,3,9-trimethyl-6H-thieno[3,2-f[]8 1,2,4]triazolo[4,3-a][1,4]diazepin-6-yl)acetamido)hexyl)amino)-2-oxoethoxy)phenyl)-3-(3,4-dimethoxyphenyl)propyl (S)-1-((S)-2-(3,4,5-trimethoxyphenyl)butanoyl)piperidine- 2-carboxylate (OGTAC-4)

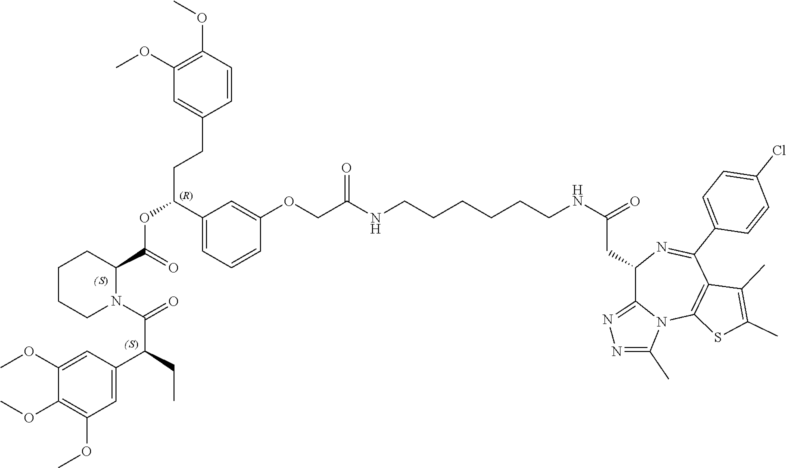

To a RBF, JQ-1 (carboxylic acid, MedChemExpress, HY-78695) (13.2 mg, 0.03 mmol) was added and dissolved in 1 mL dry DMF. Then DIPEA (26 μL) and HATU (17 mg) were added to the solution and stirred for 15 mins. AP1867-C6-NHBoc (20 mg, 0.03 mmol) was then dissolved in 1 mL dry DMF and added. The reaction was stirred for another 16 h. The reaction was then partitioned in EtOAc and H₂O, extracted with EtOAc and the dried over Na₂SO₄. The crude product was purified by PTLC (MeOH: DCM=1:30) to have white solid (28 mg, 79%). ESI-MS m/z: 1175.5 [M+H]⁺, 1196.5 [M+Na]⁺. ¹H-NMR: ¹H-NMR: δ 7.39 (d, J=8.4 Hz, 2H), 7.31 (d, J=8.4 Hz, 2H), 7.21-7.14 (m, 1H), 6.83-6.77 (m, 3H), 6.72-6.63 (m, 3H), 6.43 (d, J=2.0 Hz, 2H), 5.67 (dd, J=8.2, 5.5 Hz, 1H), 5.49-5.45 (m, 1H), 4.63 (t, 1H), 4.50 (m, 2H), 3.88-3.81 (m, 10H), 3.78 (s, 3H), 3.68 (s, 5H), 3.61 (t, J=6.7 Hz, 1H), 3.39-3.30 (m, 2H), 2.86-2.76 (t, 1H), 2.68 (s, 3H), 2.65-2.47 (m, 2H), 2.42 (s, 3H), 2.37-2.28 (m, 1H), 2.15-1.98 (m, 5H), 1.79-1.25 (m, 6H), 1.68 (s, 3H), 1.81-1.31 (m, 8H), 0.93 (t, 3H); ¹³C-NMR: δ 172.86, 170.71, 168.19, 164.65, 157.44, 153.27, 150.15, 148.95, 147.43, 142.39, 136.68, 135.41,133.47, 132.03, 131.75, 131.30, 130.64, 130.20, 129.90, 128.92, 120.29, 119.86, 113.69, 113.11, 111.79, 111.70, 105.05, 76.9, 70.64, 60.99, 56.39, 56.06, 55.96, 54.41, 52.16, 51.31, 43.57, 38.95, 38.90,38.31, 32.39, 31.38, 29.55, 28.48, 28.45, 26.88, 25.41, 21.00, 13.26, 12.82.

### VI. REFERENCES

(1) Slawson, C.; Hart, G. W. O-GlcNAc Signalling: Implications for Cancer Cell Biology. Nat. Rev. Cancer 2011, 11 (9), 678-684.
(2) Yang, X.; Qian, K. Protein O-GlcNAcylation: Emerging Mechanisms and Functions. Nat. Rev. Mol. Cell Biol. 2017, 18 (7), 452-465.
(3) Eustice, M.; Bond, M. R.; Hanover, J. A. O-GlcNAc Cycling and the Regulation of Nucleocytoplasmic Dynamics. Biochem. Soc. Trans. 2017, 45 (2), 427-436. https://doi.org/10.1042/BST20160171.
(4) Hart, G. W. Nutrient Regulation of Signaling and Transcription. J. Biol. Chem. 2019, 294 (7), 2211-2231.
(5) Ferrer, C. M.; Sodi, V. L.; Reginato, M. J. O-GlcNAcylation in Cancer Biology: Linking Metabolism and Signaling. J. Mol. Biol. 2016, 428 (16), 3282-3294.
(6) Hanover, J. A.; Chen, W.; Bond, M. R. O-GlcNAc in Cancer: An Oncometabolism-Fueled Vicious Cycle. J. Bioenerg. Biomembr. 2018, 50 (3), 155-173.
(7) Yi, W.; Clark, P. M.; Mason, D. E.; Keenan, M. C.; Hill, C.; William, A.; Iii, G.; Peters, E. C.; Driggers, E. M.; Hsieh-wilson, L. C. Phosphofructokinase 1 Glycosylation Regulates Cell Growth and Metabolism. Science (80-.). 2012, 337 (6097), 975-980.
(8) De Queiroz, R. M.; Madan, R.; Chien, J.; Dias, W. B.; Slawson, C. Changes in O-Linked N-Acetylglucosamine (O-GlcNAc) Homeostasis Activate the P53 Pathway in Ovarian Cancer Cells. J. Biol. Chem. 2016, 291 (36), 18897-18914.
(9) Gong, C. X.; Liu, F.; Iqbal, K. O-GlcNAcylation: A Regulator of Tau Pathology and Neurodegeneration. Alzheimer's Dement. 2016, 12 (10), 1078-1089.
(10) Darley-Usmar, V. M.; Ball, L. E.; Chatham, J. C. Protein O-Linked β-N-Acetylglucosamine: A Novel Effector of Cardiomyocyte Metabolism and Function. J. Mol. Cell. Cardiol. 2012, 52 (3), 538-549.
(11) Li, Y.; Xie, M.; Men, L.; Du, J. O-GlcNAcylation in Immunity and Inflammation: An Intricate System (Review). Int. J. Mol. Med. 2019, 44 (2), 363-374.
(12) Park, K.; Saudek, C. D.; Hart, G. W. Increased Expression of β-N-Acetylglucosaminidase in Erythrocytes from Individuals with Pre-Diabetes and Diabetes. Diabetes 2010, 59 (7), 1845-1850.
(13) Gorelik, A.; Bartual, S. G.; Borodkin, V. S.; Varghese, J.; Ferenbach, A. T.; van Aalten, D. M. F. Genetic Recoding to Dissect the Roles of Site-Specific Protein O-GlcNAcylation. Nat. Struct. Mol. Biol. 2019, 26 (11), 1071-1077.
(14) Wu, J. L.; Chen, Y. J.; Wu, H. Y.; Tsai, D. Y.; Chiang, M. F.; Chen, Y. J.; Gao, S.; Lin, C. C.; Lin, C. H.; Khoo, K. H.; Lin, K. I. Temporal Regulation of Lsp1 O-GlcNAcylation and Phosphorylation during Apoptosis of Activated B Cells. Nat. Commun. 2016, 7.
(15) Ramirez, D. H.; Aonbangkhen, C.; Wu, H. Y.; Naftaly, J. A.; Tang, S.; O'Meara, T. R.; Woo, C. M. Engineering a Proximity-Directed O-GlcNAc Transferase for Selective Protein O-GlcNAcylation in Cells. ACS Chem. Biol. 2020, 15 (4), 1059-1066.
(16) Schwein, P. A.; Ge, Y.; Yang, B.; D'Souza, A.; Mody, A.; Shen, D.; Woo, C. M. Writing and Erasing O-GlcNAc on Casein Kinase 2 Alpha Alters the Phosphoproteome. ACS Chem. Biol. 2022, 17, 1111-1121.
(17) Tarrant, M. K.; Rho, H. S.; Xie, Z.; Jiang, Y. L.; Gross, C.; Culhane, J. C.; Yan, G.; Qian, J.; Ichikawa, Y.; Matsuoka, T.; Zachara, N.; Etzkorn, F. A.; Hart, G. W.; Jeong, J. S.; Blackshaw, S.; Zhu, H.; Cole, P. A. Regulation of CK2 by Phosphorylation and O-GlcNAcylation Revealed by Semisynthesis. Nat. Chem. Biol. 2012, 8 (3), 262-269.
(18) Saha, A.; Bello, D.; Fernández-Tejada, A. Advances in Chemical Probing of Protein O-GlcNAc Glycosylation: Structural Role and Molecular Mechanisms. Chem. Soc. Rev. 2021, No. September 2020.
(19) Martin, S. E. S.; Tan, Z. W.; Itkonen, H. M.; Duveau, D. Y.; Paulo, J. A.; Janetzko, J.; Boutz, P. L.; Törk, L.; Moss, F. A.; Thomas, C. J.; Gygi, S. P.; Lazarus, M. B.; Walker, S. Structure-Based Evolution of Low Nanomolar O-GlcNAc Transferase Inhibitors. J. Am. Chem. Soc. 2018, 140 (42), 13542-13545.
(20) Decourcelle, A.; Loison, I.; Baldini, S.; Leprince, D.; Dehennaut, V. Evidence of a Compensatory Regulation of Colonic O-GlcNAc Transferase and O-GlcNAcase Expression in Response to Disruption of O-GlcNAc Homeostasis. Biochem. Biophys. Res. Commun. 2020, 521 (1), 125-130.
(21) Chen, P.; Hu, Z.; An, E.; Okeke, I.; Zheng, S.; Luo, X.; Gong, A.; Jaime-figueroa, S.; Crews, C. M. Modulation of Phosphoprotein Activity by Phosphorylation Targeting Chimeras (PhosTACs). 2021.
(22) Hu, Z.; Chen, P.; Li, W.; Douglas, T.; Hines, J.; Liu, Y.; Crews, C. M. Targeted Dephosphorylation of Tau by Phosphorylation Targeting Chimeras (PhosTACs) as a Therapeutic Modality. 2022.
(23) Wang, W. W.; Chen, L. Y.; Wozniak, J. M.; Jadhav, A. M.; Anderson, H.; Malone, T. E.; Parker, C. G. Targeted Protein Acetylation in Cells Using Heterobifunctional Molecules. J. Am. Chem. Soc. 2021, 143 (40), 16700-16708.
(24) Liu, N.; Ling, R.; Tang, X.; Yu, Y.; Zhou, Y.; Chen, D. Post-Translational Modifications of BRD4: Therapeutic Targets for Tumor. Front. Oncol. 2022, 12 (March), 1-11.
(25) Schwein, P. A.; Woo, C. M. The O-GlcNAc Modification on Kinases. ACS Chem. Biol. 2020, 15 (3), 602-617.
(26) Darabedian, N.; Thompson, J. W.; Chuh, K. N.; Hsieh-Wilson, L. C.; Pratt, M. R. Optimization of Chemoenzymatic Mass Tagging by Strain-Promoted Cycloaddition (SPAAC) for the Determination of O-GlcNAc Stoichiometry by Western Blotting. Biochemistry 2018, 57 (40), 5769-5774.
(27) Clackson, T.; Yang, W.; Rozamus, L. W.; Hatada, M.; Amara, J. F.; Rollins, C. T.; Stevenson, L. F.; Magari, S. R.; Wood, S. A.; Courage, N. L.; Lu, X.; Cerasoli, F.; Gilman, M.; Holt, D. A. Redesigning an FKBP-Ligand Interface to Generate Chemical Dimerizers with Novel Specificity. Proc. Natl. Acad. Sci. U. S. A. 1998, 95 (18), 10437-10442.
(28) Filippakopoulos, P.; Qi, J.; Picaud, S.; Shen, Y.; Smith, W. B.; Fedorov, O.; Morse, E. M.; Keates, T.; Hickman, T. T.; Felletar, I.; Philpott, M.; Munro, S.; McKeown, M. R.; Wang, Y.; Christie, A. L.; West, N.; Cameron, M. J.; Schwartz, B.; Heightman, T. D.; La Thangue, N.; French, C. A.; Wiest, O.; Kung. A. L.; Knapp, S.; Bradner, J. E. Selective Inhibition of BET Bromodomains. Nature 2010, 468 (7327), 1067-1073.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A method of labeling a protein of interest (POI) with a glycan, comprising:
(a) contacting, in a sample, a first fusion protein comprising a glycan transferase fused to a first proximity inducing protein, and a second fusion protein comprising the POI fused to a second proximity inducing protein, with a proximity inducing ligand; and
(b) incubating the sample for a duration sufficient for the first and second proximity inducing proteins to both to bind to the proximity inducing ligand and for the glycan transferase to label the POI with a glycan.

2. The method of claim 1, wherein the glycan transferase is a O-GlcNAc transferase, a polypeptide N-Acetylgalactosaminyltransferase 14 (GALNT14), or a glucuronosyltransferase.

3. The method of claim 1, wherein the first proximity inducing protein is a FKBP12^{F36V}.

4. The method of claim l, wherein the second proximity inducing protein is a Halotag.

5. The method of claim l, wherein the second proximity inducing protein is fused to the N-terminus or the C-terminus of the POI.

6. The method of claim 1, wherein the proximity inducing ligand comprises a first portion that is recognized and bound by the first proximity inducing protein and a second portion that is recognized and bound by the second proximity protein.

7. (canceled)

8. The method of claim 1, wherein the second portion is a haloalkane.

9. (canceled)

10. The method of claim 6, wherein the proximity inducing ligand further comprises a chemical linker between the first portion and the second portion.

11. (canceled)

12. The method of claim 1, wherein the proximity inducing ligand is: 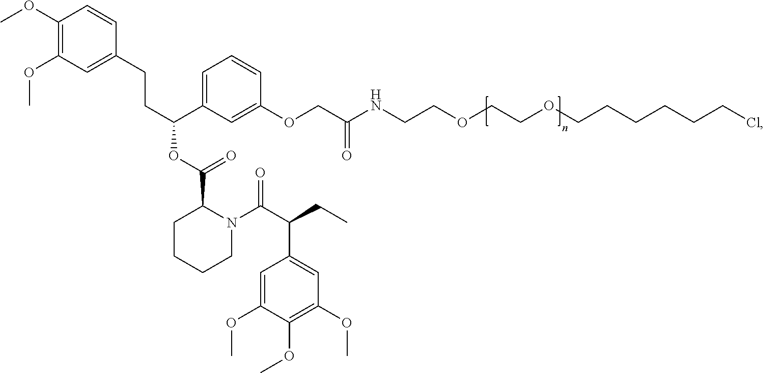
wherein n is an integer between 1 and 8.

13. (canceled)

14. The method of claim 1, further comprising the step (c) of contacting the sample with a chase ligand that is recognized and bound by the second proximity protein.

15. The method of claim 14, wherein the chase ligand comprises a detectable portion and a portion that is recognized and bound by the second proximity protein.

16. The method of claim 14, wherein the chase ligand is:

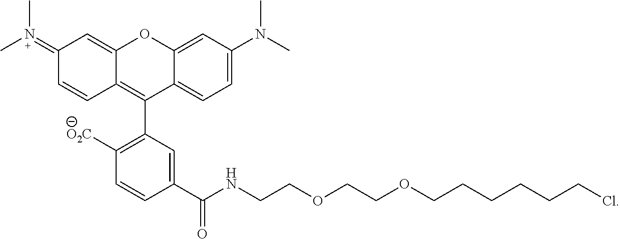

17. A method of labeling a protein of interest (POI) with a glycan, comprising:
(a) contacting, in a sample, a fusion protein comprising a glycan transferase fused to a proximity inducing protein, and the POI, with a proximity inducing ligand; and
(b) incubating the sample for a duration sufficient for the fusion protein and the POI to both to bind to the proximity inducing ligand and for the glycan transferase to label the POI with a glycan.

18. The method of claim 17, wherein the glycan transferase is a O-GlcNAc transferase, a polypeptide N-Acetylgalactosaminyltransferase 14 (GALNT14), or a glucuronosyltransferase.

19. The method of claim 17, wherein the proximity inducing protein is a FKBP12^{F36V}.

20. The method of claim 17, wherein the proximity inducing ligand comprises a first portion that is recognized and bound by the proximity inducing protein and a second portion that is recognized and bound by the POI.

21. (canceled)

22. The method of claim 20, wherein the second portion is an optionally substituted BET bromodomain inhibitor (JQ1), an optionally substituted EZH2 inhibitor, an optionally substituted BRD inhibitor, or an optionally substituted kinase inhibitor.

23. (canceled)

24. The method of claim 20, wherein the proximity inducing ligand further comprises a chemical linker between the first portion and the second portion.

25. (canceled)

26. The method of claim 17, wherein the proximity inducing ligand is:

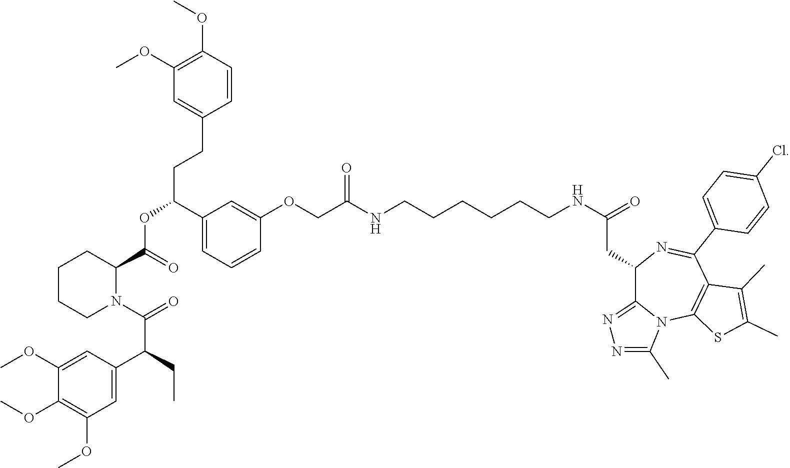

27. The method of claim 17, further comprising the step (c) of contacting the sample with a chase ligand that is recognized and bound by the POI.

28-30. (canceled)
